SEQUENCE LISTING (205 sequences)

SEQ ID NO: 1 - DNA, 20 bp, Artificial Sequence
  Quantify total Oct4 expression (forward primer)
tattcagccaaacgaccatc

SEQ ID NO: 2 - DNA, 18 bp, Artificial Sequence
  Quantify total Oct4 expression (reverse primer)
gcctctcactcggttctc

SEQ ID NO: 3 - DNA, 25 bp, Artificial Sequence
  Quantify total Sox2 expression (forward primer)
acgacgtgagcgccctgcagtacaa

SEQ ID NO: 4 - DNA, 25 bp, Artificial Sequence
  Quantify total Sox2 expression (reverse primer)
gctggagctggcctcggacttgacc

SEQ ID NO: 5 - DNA, 21 bp, Artificial Sequence
  Quantify total Klf4 expression (forward primer)
cctacacaaagagttcccatc

SEQ ID NO: 6 - DNA, 19 bp, Artificial Sequence
  Quantify total Klf4 expression reverse primer)
agtgcctggtcagttcatc

SEQ ID NO: 7 - DNA, 21 bp, Artificial Sequence
  Quantify total c-Myc expression (forward primer)
aggaacaagaagatgaggaag

SEQ ID NO: 8 - DNA, 18 bp, Artificial Sequence
  Quantify total c-Myc expression (reverse primer)
tgcgtagttgtgctgatg

SEQ ID NO: 9 - DNA, 20 bp, Artificial Sequence
  Quantify exogenous Oct4 expression (forward primer)
gtactcctcggtccctttcc

SEQ ID NO: 10 - DNA, 20 bp, Artificial Sequence
  Quantify exogenous Oct4 expression (reverse primer)
cacctgcaagtttcagcaaa

SEQ ID NO: 11 - DNA, 20 bp, Artificial Sequence
  Quantify exogenous Sox2 expression (forward primer)
catgtcccagcactaccaga

SEQ ID NO: 12 - DNA, 20 bp, Artificial Sequence
  Quantify exogenous Sox2 expression (reverse primer)
acatcccctgcttgtttcaa

SEQ ID NO: 13 - DNA, 20 bp, Artificial Sequence
  Quantify exogenous Klf4 expression (forward primer)
gaccacctcgccttacacat

SEQ ID NO: 14 - DNA, 20 bp, Artificial Sequence
  Quantify exogenous Klf4 expression (reverse primer)
ccaaaagacggcaatatggt

SEQ ID NO: 15 - DNA, 20 bp, Artificial Sequence
  Quantify exogenous c-Myc expression (forward primer)
aagaggacttgttgcggaaa

SEQ ID NO: 16 - DNA, 20 bp, Artificial Sequence
  Quantify exogenous c-Myc expression (reverse primer)
ggcattaaagcagcgtatcc

SEQ ID NO: 17 - DNA, 21 bp, Artificial Sequence
  Quantify endogenous Oct4 expression (forward primer)
aaggaattgggaacacaaagg

SEQ ID NO: 18 - DNA, 22 bp, Artificial Sequence
  Quantify endogenous Oct4 expression (reverse primer)
caagagcatcattgaacttcac

SEQ ID NO: 19 - DNA, 26 bp, Artificial Sequence
  Quantify endogenous Sox2 expression (fowrard primer)
gggaaatgggaggggtgcaaaagagg

SEQ ID NO: 20 - DNA, 26 bp, Artificial Sequence
  Quantify endogenous Sox2 expression (reverse primer)
ttgcgtgagtgtggatgggattggtg

SEQ ID NO: 21 - DNA, 19 bp, Artificial Sequence
  Quantify endogenous Klf4 expression (forward primer)
tggtgcttggtgagtcttg

SEQ ID NO: 22 - DNA, 23 bp, Artificial Sequence
  Quantify endogenous Klf4 expression (reverse primer)
aggtcataaatgttgatcggaag

SEQ ID NO: 23 - DNA, 20 bp, Artificial Sequence
  Quantify endogenous c-Myc expression (forward primer)
ccttgccgcatccacgaaac

SEQ ID NO: 24 - DNA, 23 bp, Artificial Sequence
  Quantify endogenous c-Myc expression (reverse primer)
ccttgctcgggtgttgtaagttc

SEQ ID NO: 25 - DNA, 34 bp, Artificial Sequence
  Adaptor sequence bearing EcoRI-AscI-SbfI restriction sites
acagaattcctgcgcgccaagttacggcgcgccc

SEQ ID NO: 26 - DNA, 26 bp, Artificial Sequence
  Adaptor sequence bearing EcoRI-AscI-SbfI restriction sites
ttagcatacattatacctgcaggcac

SEQ ID NO: 27 - DNA, 24 bp, Artificial Sequence
  Check for site-specific integration of OKSM-eGFP donor at AAVS1 locus (forward primer)
gctacgtcccttcggccctcaatc

SEQ ID NO: 28 - DNA, 25 bp, Artificial Sequence
  Check for site-specific integration of OKSM-eGFP donor at AAVS1 locus (reverse primer)
gcctccctaagacccagaagtccag

SEQ ID NO: 29 - DNA, 27 bp, Artificial Sequence
  Probe design for Southern blot to detect multiple integration of OKSM-eGFP cassette in genome (forward primer)
gactggtattcttaactatgttgctcc

SEQ ID NO: 30 - DNA, 24 bp, Artificial Sequence
  Probe design for Southern blot to detect multiple integration of OKSM-eGFP cassette in genome (reverse primer)
caaagggagatccgactcgtctga

SEQ ID NO: 31 - DNA, 30 bp, Artificial Sequence
  Amplify bisulfide-treated Oct3/4 promoter (forward primer)
gaggttggagtagaaggattgttttggttt

SEQ ID NO: 32 - DNA, 30 bp, Artificial Sequence
  Amplify bisulfide-treated Oct3/4 promoter (reverse primer)
cccccctaacccatcacctccaccacctaa

SEQ ID NO: 33 - DNA, 30 bp, Artificial Sequence
  Amplify bisulfide untreated Oct3/4 promoter (forward primer)
gaggctggagcagaaggattgctttggccc

SEQ ID NO: 34 - DNA, 30 bp, Artificial Sequence
  Amplify bisulfide untreated Oct3/4 promoter (reverse primer)
cccccctggcccatcacctccaccacctgg

SEQ ID NO: 35 - DNA, 25 bp, Artificial Sequence
  Amplify bisulfide-treated Nanog promoter (forward primer)
tggttaggttggttttaaatttttg

SEQ ID NO: 36 - DNA, 26 bp, Artificial Sequence
  Amplify bisulfide-treated Nanog promoter (reverse primer)
aacccacccttataaattctcaatta

SEQ ID NO: 37 - DNA, 25 bp, Artificial Sequence
  Amplify bisulfide untreated Nanog promoter (forward primer)
tggccaggctggtttcaaactcctg

SEQ ID NO: 38 - DNA, 26 bp, Artificial Sequence
  Amplify bisulfide untreated Nanog promoter (reverse primer)
gacccacccttgtgaattctcagtta

SEQ ID NO: 39 - DNA, 30 bp, Artificial Sequence
  Amplify bisulfide-treated Rex1 promoter (forward primer)
ggtttaaaagggtaaatgtgattatattta

SEQ ID NO: 40 - DNA, 22 bp, Artificial Sequence
  Amplify bisulfide-treated Rex1 promoter (reverse primer)
caaactacaaccacccatcaac

SEQ ID NO: 41 - DNA, 30 bp, Artificial Sequence
  Amplify bisulfide untreated Rex1 promoter (forward primer)
ggcctaaaagggtaaatgtgattacaccca

SEQ ID NO: 42 - DNA, 22 bp, Artificial Sequence
  Amplify bisulfide untreated Rex1 promoter (reverse primer)
caggctacagccacccatcagc

SEQ ID NO: 43 - DNA, 23 bp, Artificial Sequence
  Amplify mRNA CD133 (forward primer)
cttcatccacagatgctcctaag

SEQ ID NO: 44 - DNA, 24 bp, Artificial Sequence
  Amplify mRNA CD133 (reverse primer)
tggattcatatgccttctgtaaga

SEQ ID NO: 45 - DNA, 22 bp, Artificial Sequence
  Amplify mRNA CD44 (forward primer)
agggatcctccagctcctttcg

SEQ ID NO: 46 - DNA, 22 bp, Artificial Sequence
  Amplify mRNA CD44 (reverse primer)
cgtccgagagatgctgtagcga

SEQ ID NO: 47 - DNA, 22 bp, Artificial Sequence
  Amplify mRNA CD166 (forward primer)
gtgtgtctgggagaagacgctg

SEQ ID NO: 48 - DNA, 24 bp, Artificial Sequence
  Amplify mRNA CD166 (reverse primer)
ggtacgtcaagtcggcaaggtatg

SEQ ID NO: 49 - DNA, 23 bp, Artificial Sequence
  Amplify mRNA ABCB5 (forward primer)
atccacaagccagactagaaggc

SEQ ID NO: 50 - DNA, 24 bp, Artificial Sequence
  Amplify mRNA ABCB5 (reverse primer)
agatccaactgcttcctttctcag

SEQ ID NO: 51 - DNA, 22 bp, Artificial Sequence
  Amplify mRNA ALDH1A1 (forward primer)
tgagccagtcacctgtgttcca

SEQ ID NO: 52 - DNA, 22 bp, Artificial Sequence
  Amplify mRNA ALDH1A1 (reverse primer)
tggcagagctcctcctcagttg

SEQ ID NO: 53 - DNA, 20 bp, Artificial Sequence
  Amplify mRNA CD29 (forward primer)
atcagacgcgcagaggaggc

SEQ ID NO: 54 - DNA, 26 bp, Artificial Sequence
  Amplify mRNA CD29 (reverse primer)
gagcaaacacacagcaaactgaactg

SEQ ID NO: 55 - DNA, 22 bp, Artificial Sequence
  Amplify mRNA CTNNB1(forward primer)
acggaggaaggtctgaggagca

SEQ ID NO: 56 - DNA, 23 bp, Artificial Sequence
  Amplify mRNA CTNNB1 (reverse primer)
tgagtagccattgtccacgctgg

SEQ ID NO: 57 - DNA, 22 bp, Artificial Sequence
  Amplify mRNA LGR5 (forward primer)
acagtgcggcagacgtaaggat

SEQ ID NO: 58 - DNA, 27 bp, Artificial Sequence
  Amplify mRNA LGR5 (reverse primer)
ggagcagctgactgatgttgttcatac

SEQ ID NO: 59 - DNA, 21 bp, Artificial Sequence
  Amplify mRNA CD26 (forward primer)
gtggaaggttcttctgggact

SEQ ID NO: 60 - DNA, 24 bp, Artificial Sequence
  Amplify mRNA CD26 (reverse primer)
actgcccatcaggagatattgaat

SEQ ID NO: 61 - DNA, 20 bp, Artificial Sequence
  Amplify mRNA E-cadherin (forward primer)
gggtgactacaaaatcaatc

SEQ ID NO: 62 - DNA, 20 bp, Artificial Sequence
  Amplify mRNA E-cadherin (reverse primer)
gggggcagtaagggctcttt

SEQ ID NO: 63 - DNA, 21 bp, Artificial Sequence
  Amplify mRNA Vimentin (forward primer)
tgaaggaggaaatggctcgtc

SEQ ID NO: 64 - DNA, 23 bp, Artificial Sequence
  Amplify mRNA Vimentin (reverse primer)
gtttggaagaggcagagaaatcc

SEQ ID NO: 65 - DNA, 18 bp, Artificial Sequence
  Amplify mRNA Fibronectin (forward primer)
ggagtttcctgagggttt

SEQ ID NO: 66 - DNA, 18 bp, Artificial Sequence
  Amplify mRNA Fibronectin (reverse primer)
gcagaagtgtttgggtga

SEQ ID NO: 67 - DNA, 22 bp, Artificial Sequence
  Amplify mRNA N-cadherin (forward primer)
cgaatggatgaaagacccatcc

SEQ ID NO: 68 - DNA, 23 bp, Artificial Sequence
  Amplify mRNA N-cadherin (reverse primer)
ggagccactgccttcatagtcaa

SEQ ID NO: 69 - DNA, 24 bp, Artificial Sequence
  Amplify mRNA Snail (forward primer)
atcggaagcctaactacagcgagc

SEQ ID NO: 70 - DNA, 22 bp, Artificial Sequence
  Amplify mRNA Snail (reverse primer)
cagagtcccagatgagcattgg

SEQ ID NO: 71 - DNA, 20 bp, Artificial Sequence
  Amplify mRNA Twist (fowrard primer)
ggagtccgcagtcttacgag

SEQ ID NO: 72 - DNA, 20 bp, Artificial Sequence
  Amplify mRNA Twist (reverse primer)
tctggaggacctggtagagg

SEQ ID NO: 73 - DNA, 20 bp, Artificial Sequence
  Amplify mRNA ZEB1 (forward primer)
acccttgaaagtgatccagc

SEQ ID NO: 74 - DNA, 22 bp, Artificial Sequence
  Amplify mRNA ZEB1 (reverse primer)
cattccattttctgtcttccgc

SEQ ID NO: 75 - DNA, 20 bp, Artificial Sequence
  Amplify mRNA Slug (fowrard primer)
agatgcatattcggacccac

SEQ ID NO: 76 - DNA, 20 bp, Artificial Sequence
  Amplify mRNA Slug (reverse primer)
cctcatgtttgtgcaggaga

SEQ ID NO: 77 - DNA, 20 bp, Artificial Sequence
  Amplify mRNA TGFb1 (forward primer)
attcctggcgatacctcagc

SEQ ID NO: 78 - DNA, 20 bp, Artificial Sequence
  Amplify mRNA TGFb1 (reverse primer)
acccgttgatgtccacttgc

SEQ ID NO: 79 - DNA, 20 bp, Artificial Sequence
  Amplify mRNA Sox4 (forward primer)
ggcctcgagctgggaatcgc

SEQ ID NO: 80 - DNA, 20 bp, Artificial Sequence
  Amplify mRNA Sox4 (reverese primer)
gcccactcggggtcttgcac

SEQ ID NO: 81 - DNA, 22 bp, Artificial Sequence
  Amplify mRNA CEA (forward primer)
aacttctcctggtctctcagct

SEQ ID NO: 82 - DNA, 21 bp, Artificial Sequence
  Amplify mRNA CEA (reverse primer)
gcaaatgctttaaggaagaag

SEQ ID NO: 83 - DNA, 20 bp, Artificial Sequence
  Amplify mRNA B3GALT5 (CA 19-9) (forward primer)
caaaccaagcccagaacctg

SEQ ID NO: 84 - DNA, 22 bp, Artificial Sequence
  Amplify mRNA B3GALT5 (CA 19-9) (reverse primer)
tcaatctcatcttcgggaaagc

SEQ ID NO: 85 - DNA, 20 bp, Artificial Sequence
  Amplify mRNA ST6GALNAC6 (CA 19-9) (forward primer)
gccggagatgaggaaactga

SEQ ID NO: 86 - DNA, 20 bp, Artificial Sequence
  Amplify mRNA ST6GALNAC6 (CA 19-9) (reverse primer)
gatcacgaacactgctgacc

SEQ ID NO: 87 - DNA, 20 bp, Artificial Sequence
  Amplify mRNA TS (forward primer)
acctgaatcacatcgagcca

SEQ ID NO: 88 - DNA, 20 bp, Artificial Sequence
  Amplify mRNA TS (reverse primer)
ttggatgcggattgtaccct

SEQ ID NO: 89 - DNA, 17 bp, Artificial Sequence
  Amplify mRNA TP (forward primer)
cgcctggtgacttctcc

SEQ ID NO: 90 - DNA, 17 bp, Artificial Sequence
  Amplify mRNA TP (reverse primer)
tgggtcagcaccgaggt

SEQ ID NO: 91 - DNA, 20 bp, Artificial Sequence
  Amplify mRNA DPD (forward primer)
gttgtggctatgattgatga

SEQ ID NO: 92 - DNA, 20 bp, Artificial Sequence
  Amplify mRNA DPD (reverse primer)
attcacagataagggtacgc

SEQ ID NO: 93 - DNA, 22 bp, Artificial Sequence
  Amplify mRNA DCC (foward primer)
ttccgccatggtttttaaatca

SEQ ID NO: 94 - DNA, 21 bp, Artificial Sequence
  Amplify mRNA DCC (reverse primer)
agcctcattttcagccacaca

SEQ ID NO: 95 - DNA, 20 bp, Artificial Sequence
  Amplify mRNA KRAS (forward primer)
actggggagggctttctttg

SEQ ID NO: 96 - DNA, 21 bp, Artificial Sequence
  Amplify mRNA KRAS (reverse primer)
ggcatcatcaacaccctgtct

SEQ ID NO: 97 - DNA, 19 bp, Artificial Sequence
  Amplify mRNA TP53 (forward primer)
ggtggtgccctatgagccg

SEQ ID NO: 98 - DNA, 19 bp, Artificial Sequence
  Amplify mRNA TP53 (reverse primer)
tcctctgtgcgccggtctc

SEQ ID NO: 99 - DNA, 21 bp, Artificial Sequence
  Amplify mRNA GPA33 (forward primer)
ccaatcaaaggagggctcacc

SEQ ID NO: 100 - DNA, 22 bp, Artificial Sequence
  Amplify mRNA GPA33 (reverse primer)
ttctcttagctgctctggtggc

SEQ ID NO: 101 - DNA, 20 bp, Artificial Sequence
  Amplify mRNA BMI-1 (forward primer)
tggctcgcattcattttctg

SEQ ID NO: 102 - DNA, 20 bp, Artificial Sequence
  Amplify mRNA BMI-1 (reverse primer)
agtagtggtctggtcttgtg

SEQ ID NO: 103 - DNA, 21 bp, Artificial Sequence
  Amplify mRNA APC (forward primer)
ggaagcagagaaagtactgga

SEQ ID NO: 104 - DNA, 22 bp, Artificial Sequence
  Amplify mRNA APC (reverse primer)
ctgaagttgagcgtaataccag

SEQ ID NO: 105 - DNA, 22 bp, Artificial Sequence
  Amplify mRNA SMAD4 (forward primer)
ctgctgctggaattggtgttga

SEQ ID NO: 106 - DNA, 20 bp, Artificial Sequence
  Amplify mRNA SMAD4 (reverse primer)
ctggagggcccggtgtaagt

SEQ ID NO: 107 - DNA, 22 bp, Artificial Sequence
  Amplify mRNA AXIN2 (forward primer)
ctggctccagaagatcacaaag

SEQ ID NO: 108 - DNA, 21 bp, Artificial Sequence
  Amplify mRNA AXIN2 (reverse primer)
atctcctcaaacaccgctcca

SEQ ID NO: 109 - DNA, 22 bp, Artificial Sequence
  Amplify mRNA POLD1 (forward primer)
gactacacgggagccactgtca

SEQ ID NO: 110 - DNA, 22 bp, Artificial Sequence
  Amplify mRNA POLD1 (reverse primer)
gtaacacaggttgtgggccatc

SEQ ID NO: 111 - DNA, 20 bp, Artificial Sequence
  Amplify mRNA STK11 (forward primer)
gaagtcggaacacaaggaag

SEQ ID NO: 112 - DNA, 20 bp, Artificial Sequence
  Amplify mRNA STK11 (reverse primer)
ccgtaacctcctcagtagtt

SEQ ID NO: 113 - DNA, 22 bp, Artificial Sequence
  Amplify mRNA BMPR1A (forward primer)
gtcagaaaatggagtaacctta

SEQ ID NO: 114 - DNA, 22 bp, Artificial Sequence
  Amplify mRNA BMPR1A (reverse primer)
tagttcgctgaaccaataaagg

SEQ ID NO: 115 - DNA, 22 bp, Artificial Sequence
  Amplify mRNA MUTYH (forward primer)
ggcctctgtctccccatatcat

SEQ ID NO: 116 - DNA, 22 bp, Artificial Sequence
  Amplify mRNA MUTYH (reverse primer)
ctgctgtagggtctctgctgta

SEQ ID NO: 117 - DNA, 20 bp, Artificial Sequence
  Amplify mRNA SMAD7 (forward primer)
ctgcaacccccatcacctta

SEQ ID NO: 118 - DNA, 19 bp, Artificial Sequence
  Amplify mRNA SMAD7 (reverse primer)
ccctgtttcagcggaggaa

SEQ ID NO: 119 - DNA, 18 bp, Artificial Sequence
  Amplify mRNA BMP2 (forward primer)
ggtggaatgactggattg

SEQ ID NO: 120 - DNA, 17 bp, Artificial Sequence
  Amplify mRNA BMP2 (reverse primer)
gcatcgagatagcactg

SEQ ID NO: 121 - DNA, 20 bp, Artificial Sequence
  Amplify mRNA BMP4 (forward primer)
ctggtccaccacaatgtgac

SEQ ID NO: 122 - DNA, 20 bp, Artificial Sequence
  Amplify mRNA BMP4 (reverse primer)
cgatcggctaatcctgacat

SEQ ID NO: 123 - DNA, 22 bp, Artificial Sequence
  Amplify mRNA RHPN2 (forward primer)
gagaacgacggctactttcgga

SEQ ID NO: 124 - DNA, 22 bp, Artificial Sequence
  Amplify mRNA RHPN2 (reverse primer)
agctcttccttgagcatctgca

SEQ ID NO: 125 - DNA, 24 bp, Artificial Sequence
  Amplify mRNA SCG5 (forward primer)
atatccagctcaccaggccatgaa

SEQ ID NO: 126 - DNA, 24 bp, Artificial Sequence
  Amplify mRNA SCG5 (reverse primer)
tgttgtctccagtcaactctgcca

SEQ ID NO: 127 - DNA, 20 bp, Artificial Sequence
  Amplify mRNA GREM1 (forward primer)
gtcacactcaactgccctga

SEQ ID NO: 128 - DNA, 20 bp, Artificial Sequence
  Amplify mRNA GREM1 (reverse primer)
ggtgaggtgggtttctggta

SEQ ID NO: 129 - DNA, 22 bp, Artificial Sequence
  Amplify mRNA FMN1 (forward primer)
cagcatctctgttcctccatca

SEQ ID NO: 130 - DNA, 22 bp, Artificial Sequence
  Amplify mRNA FMN1 (reverse primer)
agtgccttccattatgcctacc

SEQ ID NO: 131 - DNA, 32 bp, Artificial Sequence
  Construct PGK-Luc-Intron to examine intron 2 (2.4kb) transcriptional regulation activity of CD24 (forward primer)
gcgctctagactgcagggattagcgcctggag

SEQ ID NO: 132 - DNA, 37 bp, Artificial Sequence
  Construct PGK-Luc-Intron to examine intron 2 (2.4kb) transcriptional regulation activity of CD24 (reverse primer)
atatggccggccccacatcacagctacctccatgtac

SEQ ID NO: 133 - DNA, 31 bp, Artificial Sequence
  Construct CD24-derived luciferase reporter to examine core promoter activity of CD24 (forward primer)
gatgatatcgataccagccgggtaccagcag

SEQ ID NO: 134 - DNA, 31 bp, Artificial Sequence
  Construct CD24-derived luciferase reporter to examine core promoter activity of CD24 (reverse primer)
gataagctttcaggatgctgggtgcttggag

SEQ ID NO: 135 - DNA, 33 bp, Artificial Sequence
  Construct CD24-derived luciferase reporter to examine core promoter activity of CD24 (forward primer)
gatgatatcgcagtctgagtggcaatgcacttg

SEQ ID NO: 136 - DNA, 31 bp, Artificial Sequence; identical to SEQ ID NO: 134
  Construct CD24-derived luciferase reporter to examine core promoter activity of CD24 (reverse primer)

SEQ ID NO: 137 - DNA, 32 bp, Artificial Sequence
  Construct CD24-derived luciferase reporter to examine core promoter activity of CD24 (forward primer)
gatgatatccctgtagtttgcagcgtcaggca

SEQ ID NO: 138 - DNA, 31 bp, Artificial Sequence; identical to SEQ ID NO: 134
  Construct CD24-derived luciferase reporter to examine core promoter activity of CD24 (reverse primer)

SEQ ID NO: 139 - DNA, 31 bp, Artificial Sequence
  Construct CD24-derived luciferase reporter to examine core promoter activity of CD24 (forward primer)
gatgatatcccacgcccggccaaagtatttc

SEQ ID NO: 140 - DNA, 31 bp, Artificial Sequence; identical to SEQ ID NO: 134
  Construct CD24-derived luciferase reporter to examine core promoter activity of CD24 (reverse primer)

SEQ ID NO: 141 - DNA, 38 bp, Artificial Sequence
  CD24 promoter region bearing a Sox2 binding site (forward primer)
tggcaggtcccgggaaacaaaggaaacttgggcccggc

SEQ ID NO: 142 - DNA, 38 bp, Artificial Sequence
  CD24 promoter region bearing a Sox2 binding site (reverse primer)
gccgggcccaagtttcctttgtttcccgggacctgcca

SEQ ID NO: 143 - DNA, 38 bp, Artificial Sequence
  CD24 promoter region bearing two Sox2 binding sites (forward primer)
ccgggaaacaaaggaaactccgggaaacaaaggaaact

SEQ ID NO: 144 - DNA, 38 bp, Artificial Sequence
  CD24 promoter region bearing two Sox2 binding sites (reverse primer)
agtttcctttgtttcccggagtttcctttgtttcccgg

SEQ ID NO: 145 - DNA, 38 bp, Artificial Sequence
  CD24 promoter region bearing no Sox2 binding site (none relevant sequence 1) (forward primer)
ccagccgggtaccagcagccggcggcgcccgcccacct

SEQ ID NO: 146 - DNA, 38 bp, Artificial Sequence
  CD24 promoter region bearing no Sox2 binding site (none relevant sequence 1) (reverse primer)
aggtgggcgggcgccgccggctgctggtacccggctgg

SEQ ID NO: 147 - DNA, 38 bp, Artificial Sequence
  CD24 promoter region bearing no Sox2 binding site (none relevant sequence 2) (forward primer)
gctttcctggtatataaggtctcgccggctcgccgcgc

SEQ ID NO: 148 - DNA, 38 bp, Artificial Sequence
  CD24 promoter region bearing no Sox2 binding site (none relevant sequence 2) (reverse primer)
gcgcggcgagccggcgagaccttatataccaggaaagc

SEQ ID NO: 149 - DNA, 38 bp, Artificial Sequence
  CD24 promoter region bearing a mutated Sox2 binding site (forward primer)
tggcaggtcccgggaaaaggaggaaacttgggcccggc

SEQ ID NO: 150 - DNA, 38 bp, Artificial Sequence
  CD24 promoter region bearing a mutated Sox2 binding site (reverse primer)
gccgggcccaagtttcctccttttcccgggacctgcca

SEQ ID NO: 151 - DNA, 38 bp, Artificial Sequence
  CD24 promoter region bearing a consensus Sox2 binding site (positive control) (forward primer)
tggcaggtccatttgcataacaaagagttgggcccggc

SEQ ID NO: 152 - DNA, 38 bp, Artificial Sequence
  CD24 promoter region bearing a consensus Sox2 binding site (positive control) (reverse primer)
gccgggcccaactctttgttatgcaaatggacctgcca

SEQ ID NO: 153 - DNA, 26 bp, Artificial Sequence
  Construct CRISPR to knockout miR205 binding site on CD24 3′-UTR (forward primer)
aaacgagtttcatgtacaagatgagt

SEQ ID NO: 154 - DNA, 26 bp, Artificial Sequence
  Construct CRISPR to knockout miR205 binding site on CD24 3′-UTR (reverse primer)
taaaactcatcttgtacatgaaactc

SEQ ID NO: 155 - DNA, 24 bp, Artificial Sequence
  T7E1 assay to quantify CRISPR-mediated cleavage efficiency on targeted CD24 site (forward primer)
cagtcaacagccagtctcttcgtg

SEQ ID NO: 156 - DNA, 23 bp, Artificial Sequence
  T7E1 assay to quantify CRISPR-mediated cleavage efficiency on targeted CD24 site (reverse primer)
catcattgccctggcacatgtca

SEQ ID NO: 157 - DNA, 34 bp, Artificial Sequence
  Construct left homologous arm bearing CD24 sequences (forward primer)
gcgctacgtaccaggatagacagtgacccaatga

SEQ ID NO: 158 - DNA, 34 bp, Artificial Sequence
  Construct left homologous arm bearing CD24 sequences (reverse primer)
atatgtcgaccttgtacatgaaactccagcagat

SEQ ID NO: 159 - DNA, 37 bp, Artificial Sequence
  Construct right homologous arm bearing CD24 sequences (forward primer)
atatgcggccgcgaaggagaggcaacatccaaaatag

SEQ ID NO: 160 - DNA, 35 bp, Artificial Sequence
  Construct right homologous arm bearing CD24 sequences (reverse primer)
gcgcttcgaatcaagcctgtaatcccagcactttg

SEQ ID NO: 161 - DNA, 25 bp, Artificial Sequence
  Check for site-specific integration of mCherry transgene on CD24 (forward primer)
gacatcacctcccacaacgaggact

SEQ ID NO: 162 - DNA, 25 bp, Artificial Sequence
  Check for site-specific integration of mCherry transgene on CD24 (reverse primer)
agcatcagtgtgtgaccatgcgaac

SEQ ID NO: 163 - DNA, 20 bp, Artificial Sequence
  Amplify mRNA CD24 on chromosome 6 (forward primer)
gagcaatggtggccaggctc

SEQ ID NO: 164 - DNA, 25 bp, Artificial Sequence
  Amplify mRNA CD24 on chromosome 6 (reverse primer)
ggattgggtttagaagatggggaaa

SEQ ID NO: 165 - DNA, 20 bp, Artificial Sequence
  Amplify mRNA CD24 on chromosome Y (forward primer)
gagcaatggtggccaggctt

SEQ ID NO: 166 - DNA, 25 bp, Artificial Sequence
  Amplify mRNA CD24 on chromosome Y (reverse primer)
ggattgggtttagaagatggggaag

SEQ ID NO: 167 - DNA, 20 bp, Artificial Sequence
  Amplify mRNA CD24 on chromosome 15 (forward primer)
atccagccagacacgctgca

SEQ ID NO: 168 - DNA, 23 bp, Artificial Sequence
  Amplify mRNA CD24 on chromosome 15 (reverse primer)
cagtagctggatttggggcagtc

SEQ ID NO: 169 - DNA, 22 bp, Artificial Sequence
  Amplify truncated mRNA CD24 expressed by chromosome 6 (forward primer)
tctggaagtccaatgtggcaag

SEQ ID NO: 170 - DNA, 25 bp, Artificial Sequence
  Amplify truncated mRNA CD24 expressed by chromosome 6 (reverse primer)
cactggaagttcccttctcatgtac

SEQ ID NO: 171 - DNA, 24 bp, Artificial Sequence
  Quantify precursor stem-loop hsa-mir-205 expression (forward primer)
agatcctcagacaatccatgtgct

SEQ ID NO: 172 - DNA, 20 bp, Artificial Sequence
  Quantify precursor stem-loop hsa-mir-205 expression (reverse primer)
agctccatgcctcctgaact

SEQ ID NO: 173 - DNA, 28 bp, Artificial Sequence
  cDNA synthesis of mature miR205-5p (adapter)
tccgtcgttctaatgcgaacagactccg

SEQ ID NO: 174 - DNA, 21 bp, Artificial Sequence
  Quantify mature miR205-5p  expression (forward primer)
cctccatccttcattccaccg

SEQ ID NO: 175 - DNA, 22 bp, Artificial Sequence
  Quantify mature miR205-5p  expression (reverse primer)
gtttccgtcgttctaatgcgaa

SEQ ID NO: 176 - DNA, 22 bp, Artificial Sequence
  Quantify miR205HG expression (forward primer)
ctcaagtacccatcttggaggg

SEQ ID NO: 177 - DNA, 21 bp, Artificial Sequence
  Quantify miR205HG expression (reverse primer)
cacgcacactccagatgtctc

SEQ ID NO: 178 - DNA, 36 bp, Artificial Sequence
  Construct CMV-miR205 (forward primer)
gcgcgaattcaaagatcctcagacaatccatgtgct

SEQ ID NO: 179 - DNA, 33 bp, Artificial Sequence
  Construct CMV-miR205 (reverse primer)
atatactagttgtcagctccatgcctcctgaac

SEQ ID NO: 180 - DNA, 32 bp, Artificial Sequence
  Construct CMV-miR205HG (co-expressing miR205) (forward primer)
gcgcgaattcgaaatgggctgagtccctcttg

SEQ ID NO: 181 - DNA, 36 bp, Artificial Sequence
  Construct CMV-miR205HG (co-expressing miR205) (reverse primer)
atatactagttttttcagtagacaagcaacttttag

SEQ ID NO: 182 - DNA, 35 bp, Artificial Sequence
  Construct PGK-Luc-UTR to examine 3′-UTR (211bp) transcriptional regulation activity of CD24 (forward primer)
gcgctctagacttaagagactcaggccaagaaacg

SEQ ID NO: 183 - DNA, 37 bp, Artificial Sequence
  Construct PGK-Luc-UTR to examine 3′-UTR (211bp) transcriptional regulation activity of CD24 (reverse primer)
atatggccggccggcatccatcatctagtcaaacctc

SEQ ID NO: 184 - DNA, 35 bp, Artificial Sequence; identical to SEQ ID NO: 182
  Construct PGK-Luc-UTR to examine 3′-UTR transcriptional regulation activity of CD24 (forward primer)

SEQ ID NO: 185 - DNA, 39 bp, Artificial Sequence
  Construct PGK-Luc-UTR to examine 3′-UTR transcriptional regulation activity of CD24 (reverse primer)
atatggccggcccaagccacattcaaggaaatcatgtct

SEQ ID NO: 186 - DNA, 26 bp, Artificial Sequence
  Construct CRISPR to knockout CD24 ORF (forward primer)
aaacggacatgggcagagcaatgggt

SEQ ID NO: 187 - DNA, 26 bp, Artificial Sequence
  Construct CRISPR to knockout CD24 ORF (reverse primer)
taaaacccattgctctgcccatgtcc

SEQ ID NO: 188 - DNA, 24 bp, Artificial Sequence
  T7E1 assay to quantify CRISPR-mediated cleavage efficiency on targeted CD24 site (forward primer)
cacgtcacggctattgtggctttc

SEQ ID NO: 189 - DNA, 25 bp, Artificial Sequence
  T7E1 assay to quantify CRISPR-mediated cleavage efficiency on targeted CD24 site (reverse primer)
gcctctgggtgaaagtgggaagtag

SEQ ID NO: 190 - DNA, 35 bp, Artificial Sequence
  Construct left homologous arm bearing CD24 sequences (forward primer)
gcgctacgtagctcacagaacaaagcaagggcttc

SEQ ID NO: 191 - DNA, 30 bp, Artificial Sequence
  Construct left homologous arm bearing CD24 sequences (reverse primer)
atatgtcgacttgctctgcccatgtcccct

SEQ ID NO: 192 - DNA, 31 bp, Artificial Sequence
  Construct right homologous arm bearing CD24 sequences (forward primer)
atatgcggccgctggtggccaggctcgggct

SEQ ID NO: 193 - DNA, 36 bp, Artificial Sequence
  Construct right homologous arm bearing CD24 sequences (reverse primer)
gcgcttcgaaaggatctagggagaccgcgctggtag

SEQ ID NO: 194 - DNA, 25 bp, Artificial Sequence
  Check for site-specific integration of mCherry transgene on CD24 (forward primer)
gacatcacctcccacaacgaggact

SEQ ID NO: 195 - DNA, 24 bp, Artificial Sequence
  Check for site-specific integration of mCherry transgene on CD24 (reverse primer)
atcctccaaacccgaactgaccca

SEQ ID NO: 196 - DNA, 20 bp, Artificial Sequence
  Amplify mRNA CD24 (fowrad primer)
gagcaatggtggccaggctc

SEQ ID NO: 197 - DNA, 25 bp, Artificial Sequence
  Amplify mRNA CD24 (reverse primer)
ggattgggtttagaagatggggaaa

SEQ ID NO: 198 - DNA, 31 bp, Artificial Sequence
  Amplify mRNA DPP4 (forward primer)
cattcacttttgaaaagttctccctagaaag

SEQ ID NO: 199 - DNA, 28 bp, Artificial Sequence
  Amplify mRNA DPP4 (reverse primer)
cgtttttgtgcagttttaaaatgtgtgc

SEQ ID NO: 200 - DNA, 28 bp, Artificial Sequence
  Amplify mRNA ITGB1 (forward primer)
ggttctcatatgaacctaactggtcaaa

SEQ ID NO: 201 - DNA, 32 bp, Artificial Sequence
  Amplify mRNA ITGB1(reverse primer)
atcatgtttataaaagcaatagaaggtacggt

SEQ ID NO: 202 - DNA, 26 bp, Artificial Sequence
  Amplify mRNA KIT (forward primer)
aaggtgatctatttttccctttctcc

SEQ ID NO: 203 - DNA, 25 bp, Artificial Sequence
  Amplify mRNA KIT (reverse primer)
tttcatactgaccaaaactcagcct

SEQ ID NO: 204 - DNA, 23 bp, Artificial Sequence
  Amplify mRNA MET (forward primer)
gactcctacaacccgaatactgc

SEQ ID NO: 205 - DNA, 26 bp, Artificial Sequence
  Amplify mRNA MET (reverse primer)
catagtgctccccaatgaaagtagag

(19) United States
(12) Patent Application Publication
(10) Pub. No.: US 2017/0191034 A1
(43) Pub. Date: Jul. 6, 2017

(54) A METHOD TO UP-REGULATE CANCER STEM CELL MARKERS FOR THE GENERATION OF ANTIGEN SPECIFIC CYTOTOXIC EFFECTOR T CELLS

(71) Applicants: AGENCY FOR SCIENCE TECHNOLOGY AND RESEARCH, Singapore (SG); TESSA THERAPEUTICS PTE. LTD., Singapore (SG)

(72) Inventors: Shu WANG, Singapore (SG); Chunxiao WU, Singapore (SG); Jieming ZENG, Singapore (SG); Yovita Ida PURWANTI, Singapore (SG); Andrew KHOO, Singapore (SG)

(21) Appl. No.: 15/508,897

(22) PCT Filed: Sep. 4, 2015
(86) PCT No.: PCT/SG2015/050301

§ 371 Date: Mar. 3, 2017

(30) Foreign Application Priority Data
Sep. 4, 2014 (SG) 10201405497U

(51) Int. Cl. C12N 5/0784; C12N 5/095

(52) U.S. Cl. CPC C12N 5/0639; C12N 5/0695; C12N 2501/602; C12N 2501/603; C12N 2501/604; C12N 2501/606; C12N 2502/30; C12N 2506/30

(57) ABSTRACT

The invention concerns a method of preparing a composition comprising stimulated immune system cells such as dendritic cells (DC) for use in inducing immune response of cytotoxic T lymphocytes against colorectal cancer. The dendritic cells are pulsed by contact with colorectal cancer stem cells (CSC) or their fragments thereof. These colorectal CSCs are produced by OSKM (Oct4, Sox2, Klf4 and c-Myc) induced reprogramming of differentiated colorectal cancer cells (CRC) which results in undifferentiated colorectal CSC-like cells. Both the CSC-like cells and the lysates of heat-shocked CSC-like cells could be used as an accessible source of tumour antigens for DC pulsing to induce specific immune responses against colorectal CSCs.

### CROSS-REFERENCE TO RELATED APPLICATIONS

This application claims the benefit of priority of Singapore Patent Application No. 102014054970, filed 4 September, 2014, the contents of it being hereby incorporated by reference in its entirety for all purposes.

### TECHNICAL FIELD

The present invention generally relates to cancer immunotherapy. In particular, the present invention relates to the activation of immune system cells against cancer cells using re-programmed colorectal cancer stem cells.

### BACKGROUND ART

Colorectal cancer (CRC) is one of the most commonly diagnosed cancers worldwide, and is the second or third leading cause of cancer-related mortality in many industrialized countries. More than 50% of the patients who are initially diagnosed with localized CRC ultimately develop stage IV CRC with distant metastasis or recurrence. It has been found that small subpopulations of highly tumorigenic (or tumor-initiating) cells, i.e. colorectal cancer stem cells (CSCs), are present in various human malignancies, such as CRCs. CSCs hold stem cell abilities to self-renew and to give rise to phenotypically diverse tumor cell populations. These tumorigenic cells are crucial in sustaining tumor growth and initiating distant metastases. CSCs are also thought to be responsible for the resistance of tumors to major conventional therapeutic strategies, such as chemotherapy and radiotherapy. The failure to fully eradicate CSCs by these conventional strategies is thought to allow for tumor relapse and metastasis, despite primary tumor removal and tumor bulk shrinkage. Therefore, CSCs are considered to be responsible for tumorigenesis, cancer recurrence, metastasis, and the failure of CRC treatment.

Novel therapeutic approaches targeting CSCs are therefore needed to prevent CRC metastasis and tumor recurrence. One approach that is being explored is cancer immunotherapy based on cancer stem cells. However, the development of this strategy is being limited by the lack of sufficient quantities of tumor cells to provide tumor-specific antigens (TSAs) and tumor-associated antigens (TAAs) for activating immune system cells against CRC cells.

It is therefore an object of the present invention to provide an improved cancer immunotherapy against colorectal cancer that overcomes, or at least ameliorates, one or more of the disadvantages described above.

It is also an object of the present invention to provide sufficient quantities of tumor cells bearing tumor-specific antigens (TSAs) and tumor-associated antigens (TAAs) to activate immune system cells against CRC cells for cancer immunotherapy.

It is yet another object of the present invention to provide sufficient quantities of antigens, such as tumor-specific antigens (TSAs) and tumor-associated antigens (TAAs), for activation of immune system cells against CRC cells for cancer immunotherapy.

### SUMMARY OF INVENTION

In a first aspect, there is provided a composition comprising at least one isolated immune system cell pulsed by contact to at least one colorectal cancer stem cell, or fragments thereof, wherein the colorectal cancer stem cell is obtained by reverting at least one differentiated colorectal tumour cell to have undifferentiated colorectal stem-cell state.

In a second aspect, there is provided a composition for stimulating the immune system of a subject comprising at least one antigen presenting cell pulsed by contact to one or more antigens obtained from a colorectal cancer stem cell, wherein the colorectal cancer stem cell is obtained by reverting at least one differentiated colorectal tumour cell to have undifferentiated colorectal stem-cell state, wherein the reversion is obtained by reprogramming the differentiated colorectal tumour cell obtained from the subject with cell reprogramming factors or transcription factors selected from the group consisting of Oct4 and Sox2.

In a third aspect, there is provided a composition for pulsing a dendritic cell such that the dendritic cell is capable of inducing specific immune response of cytotoxic T lymphocytes against an in vitro colorectal cancer stem cell, the composition comprising at least one in vitro colorectal cancer stem cell-like cell, or fragments thereof, which has been enriched from a re-programmed in vitro colorectal cancer cell.

In a fourth aspect, there is provided a method of producing a stimulated immune system cell comprising: contacting a colorectal cancer stem cell, or fragments thereof, with at least one immune system cell, wherein the colorectal cancer stem cell is obtained by reverting at least one differentiated colorectal tumour cell to its undifferentiated stem-cell state.

In a fifth aspect, there is provided a vaccine comprising at least one immune cell stimulated with the method as defined herein.

In a sixth aspect, there is provided a stimulated immune cell obtained from the method as defined herein.

In a seventh aspect, there is provided a method of treating a colorectal cancer in a subject, comprising: immunising the subject with a composition defined herein or a vaccine as defined herein or an immune cell stimulated by the methods as defined herein.

In an eighth aspect, there is provided the use of a composition as defined herein or a vaccine as defined herein or an immune cell stimulated by the methods as defined herein, in the manufacture of a medicament for treating a colorectal cancer in a subject.

In a ninth aspect, there is provided a method of producing an anti-colorectal cancer vaccine for a subject comprising autologous dendritic cells, comprising the steps of: (a) extracting and purifying peripheral blood mononuclear cells (monocytes) obtained from a sample from the subject; (b) cultivating the monocytes under conditions effective to induce monocytes to dendritic cell differentiation; (c) contacting the cultivated immature dendritic cells of (b) with a cancer stem cell, or fragment thereof, obtained by reverting at least one differentiated colorectal tumour cell to its undifferentiated colorectal stem-cell state; and (d) cultivating the colorectal cancer stem-cell loaded dendritic cells of (c) with a dendritic cell maturation-inducing agent; and (e) harvesting the colorectal cancer stem-cell loaded mature dendritic cells as the anti-cancer vaccine.

### BRIEF DESCRIPTION OF DRAWINGS

The invention will be better understood with reference to the detailed description when considered in conjunction with the non-limiting examples and the accompanying drawings, in which:

FIG. 1 shows reprogramming of colorectal cancer (CRC) cells to generate induced pluripotent cancer (iPC) cells. (A) is a schematic diagram showing AAVS1-locus integration of the OSKM expression cassette provided by the baculovirus (BV) DNA donor BV-OSKM following BV-ZFN-induced DNA DBS. HR(L) & HR(R): Left and right arms for HR. FP & RP: Binding sites for PCR forward and reverse primers used for PCR genotyping. (B) shows iPC cell generation after BV transduction. Formation of iPS cell-like colony on mitomycin C-inactivated mouse embryonic fibroblasts (MEF) was observed. The phase contrast (left) and eGFP fluorescent (right) images of a colony derived from HCT8 cells are shown. The cells were cultured in a human iPS cell medium. Results from human CRC HCT8 subclones are shown. (C) shows the results of PCR genotyping to confirm the targeted integration of the OSKM cassette into the AAVS1 locus. The amplification of a 3-kb DNA fragment as shown in (A) is used to identify the targeted insertion. (D) shows expression of pluripotency markers in iPC cells derived from HCT-8 cells. Immunostaining was performed to detect the expression of Nanog, SSEA-4 and TRA-1-60 in an iPC cell colony. (E) shows hematoxylin and eosin-staining of tissue sections of a teratoma formed by the derived iPC cells. The histology of differentiated tissues found in the teratoma demonstrates immature neuroglial tissue and neuroepithelial rosettes (ectoderm), cartilage (mesoderm), and gland (endoderm).

FIG. 2 shows ectopic expression of OSKM leads to increased CSC-like properties in human CRC cells. (A) are graphs showing OSKM gene expression quantified by RT-qPCR. Primers were designed to amplify both endogenous and exogenous OSKM genes. The GAPDH gene expression was used to normalize and calculate the fold change. (B) is a graph that illustrates tumorsphere formation efficiency. The efficiencies of two OSKM-expressing clones, HCT3.11 and SW1.9, were tested. Results were normalized to their parent cells. (C) is the result of flow cytometry analysis to quantify the percentage of CSC marker-positive cells. Results shown are mean±SD of three experiments.

FIG. 3 describes the molecular characterization of OSKM-expressing colorectal CSC-like cells by quantitative RTPCR analysis. (A) is a graph showing the results for colorectal CSC markers. (B) is a graph showing the results for epithelial-to-mesenchymal transition (EMT) markers. (C) is a graph showing the results for commonly used CRC clinical biomarkers. (D) is a graph showing the results for genes associated with CRC top risk loci identified by GWASs. The wild-type HCT-8 derived single cell clone subjected to OSKM genetic modification was used as the control. All fold changes are normalized to the GAPDH expression levels.

FIG. 4 illustrates generation of Oct4-reactive T cells from human PBMCs by priming CD8+ naïve T cells with dendritic cells loaded with lysates of OSKM gene-expressing CRC cells. (A) is a schematic diagram of the protocol: DCs were developed and CD8+ naïve T cells were selected from HLA-A2+ human PBMCs of a healthy donor. After DC pulsing with tumor lysates and DC maturation, the DCs were used to stimulate in vitro autologous T cells for two consecutive weeks. Viable T cells were then harvested and evaluated in an IFN-gamma ELISPOT assay for their antigen-specific INF-gamma responses. (B) shows DC morphology during differentiation and after pulsing with tumor lysates and maturation. (C) shows characterization of DCs by flow cytometry analysis before and after pulsing and maturation. It is seen that upon maturation the expression of CD83 and CD40 increased significantly. (D) shows characterization of naïve T cells by flow cytometry analysis before and after selection. An increase in CD8+ population and depletion of CD45RO+ and CD57+ memory T cells were observed. The graph in (E) shows DCs pulsed with HCT3.11 tumor lysates stimulated autologous Oct4-reactive T cell response in vitro. T cells were interrogated for reactivity against tumor antigens in IFN-gamma EliSpot by re-stimulation with T2 cells loaded with Oct4 and GFP (positive control) peptides. INF-gamma responses of T cells previously stimulated with unpulsed DCs, DCs pulsed with lysates of wild-type (WT) HCT8 cells, DCs pulsed with lysates of Oct4- and GFP-expressing HCT3.11 clone, T cells alone and T2 cells alone are shown. ***, p<0.001 versus T2 alone by analysis of variance.

FIG. 5 shows the effects of DC pulsing with heat-shocked tumor cells on the generation of colorectal CSC-reactive T cells. (A) shows that heat shock up-regulated Hsp70 in OSKM-expressing HCT3.11 cells without affecting Oct4 expression. Sample #1 and #2: Cells in Cellgro medium with (#1) or without (#2) heat shock. Sample #3 and #4: Cells in PBS with (#4) or without (#3) heat shock. (B) shows that DC marker expression on matured DCs (mDCs) was not affected after pulsing with the supernatants collected from heat-shocked HCT3.11 cells. DC characterization was performed with flow cytometry. (C) is the result of characterization of T cells after DC priming by flow cytometry analysis. (D) shows that DCs pulsed with the supernatants collected from heat shocked HCT3.11 cells stimulated autologous T cell response against colorectal CSCs in vitro. T cells were interrogated for reactivity against tumor antigens in IFN-gamma EliSpot by re-stimulation with T2 cells loaded with indicated peptides. T cells without DC priming, T cells stimulated with unpulsed DCs, and T cells stimulated with DCs after pulsing with whole lysates of HCT3.11 without heat shock are included for comparison.

FIG. 6 outlines the strategy to employ OSKM reprogramming of CRC cells for DC vaccination against CRC CSCs. OSKM: Oct4, Sox2, Klf4, and c-Myc; CTLs: Cytotoxic T lymphocytes.

### DESCRIPTION OF EMBODIMENTS

Cancer cell reprogramming is a reverse process of CSC differentiation into tumour cells. This process facilitates dedifferentiation of non-tumorigenic cancer cells toward a less differentiated state, resulting in a population of cells with tumour-initiating capacity.

In a first aspect, there is provided a composition comprising at least one isolated immune system cell pulsed by contact to at least one colorectal cancer stem cell, or fragments thereof, wherein the colorectal cancer stem cell is obtained by reverting at least one differentiated colorectal tumour cell to have undifferentiated colorectal stem-cell state.

The isolated immune system cell may be an antigen presenting cell. Exemplary antigen presenting cells include, but are not limited to, a dendritic cell, a macrophage, a B-cell, an activated epithelial cell, and the like.

In one example, the antigen presenting cell is a dendritic cell. Identification and reorganization of DCs as the most efficient antigen presenting cells has been one of the significant advances in cancer immunotherapy. Exogenous antigens can be captured by DC endocytosis, released to its cytoplasmic compartment, and routed to the MHC-I antigen presentation pathway. DC vaccination is based on this antigen cross-presentation process and can induce CD8+ tumor antigen-specific cytotoxic T lymphocytes (CTLs), representing a potentially potent therapeutic approach for cancer. DCs also play a critical role in bridging innate and acquired immunity by facilitating crosstalk between immune effector cells, such as conventional αβ T cells, γδ T cells, NK cells, and invariant NKT cells, directly or indirectly through cytokines derived from the effector cells and DCs.

Previous cancer immunotherapies have involved targeting cells that express differentiated tumor antigens. Recent findings from animal studies, cultured human tumor cells and clinical blood samples support the notion that DC-based CSC vaccination confers superior protective antitumor immunity by selectively targeting CSCs. DC-based CSC vaccines can elicit humoral and cellular immune responses directed towards stem cell antigens and are associated with the induction of efficient protective anti-CSC immunity. For example, DC-mediated targeting of glioma CSC neurospheres led to greater antitumor immunity in mice compared to targeting bulk tumor cells. These studies indicate that although immune tolerance to pathways shared between CSCs and normal adult stem cells is possible, the immune system can still recognize and react to the differences between the two types of stem cells, and consequently eradicate CSCs. Since CSCs have a recognized refractoriness to traditional therapies involving chemo and radiation, immune intervention as a therapeutic platform, which targets TSAs and/or TAAs and is less dependent on the metabolic or proliferative state of cancerous cells, has become particularly attractive. The resistance of CSCs to conventional therapies indicates that these cancer-initiating cells may contain numerous mutations that encode tumor-specific neoantigens, in addition to stem cell antigens. Thus, using CSCs as a source of tumor antigens for DC vaccine preparation provides a way to stimulate immune responses directed toward unidentified neoantigens and tumorigenic antigens associated with CSCs, leading to CSC killing with long-lasting benefits.

For DC cancer immunotherapy, it is important that large quantities of tumor cells are available to provide tumor-specific antigens (TSAs) and tumor-associated antigens (TAAs) for DC pulsing. CSCs isolated from primary tumors will rapidly differentiate into the non-CSCs that represent the majority of cells in tumors in vitro. This is a major limiting factor that has slowed down the discovery of drugs targeting CSCs and a technical barrier preventing large-scale production of CSCs for clinical cancer immunotherapy.

The immune system cells of the present disclosure are “isolated”. The term isolated as used herein means altered “by the hand of man” from its natural state; i.e., if it occurs in nature, it has been changed or removed from its original environment, or both. The isolated immune system cells may be pulsed by contact to at least one colorectal CSC, or fragments thereof. The isolated immune system cells may be, for example, immature dendritic cells. The term “pulsing” refers to loading of an immature dendritic cell with antigen(s), or exposing an immature dendritic cell to antigen(s), over a short period of time. Hence, “pulsing” an immature dendritic cell, optionally in the presence of one or more maturation-inducing agent as described herein, may result in maturation of an immature dendritic cell into a mature dendritic cell.

By “contact,” a cell may be in physical association with another cell, or fragments thereof. For example, an isolated immune system cell may “contact” a colorectal cancer stem cell, or cell fragment, by coming into close proximity to or by physically associating with the colorectal cancer stem cell, or fragment thereof. The contact may be direct contact, or via an intermediary agent. An immature dendritic cell may be allowed to come into contact with tumour antigens by “pulsing” it with tumour antigens, such as those present in heat-shocked tumour lysate.

Therefore in one example, the colorectal cancer stem cell may be a heat-shocked colorectal cancer stem cell, or fragments thereof. The fragment may be a lysate of a heat-shocked colorectal stem cell. Dying tumour cells provide both a source of tumour antigens and endogenous danger signals that are capable of triggering cell activation. Heat stress may therefore be used to induce tumour cell death before using their lysate, for example, for DC pulsing. Heat shock typically involves subjecting a cell to a higher temperature than that of the normal physiological temperature of the cell in the body. The process of heat shock can be done in various media, for example in a CO₂ incubator, an O₂ incubator, or in a hot water bath. For example, cells can be heat-treated at about 42° C. for about 60 min. Advantageously, the process of heat-shock of the colorectal cancer stem cell allows generation of mature dendritic cells that are more adept at eliciting CSC antigen specific CTLs, as described in Example 4 below.

In one example, the dendritic cell may be an immature dendritic cell. For example, the immature dendritic cell may be generated from peripheral blood mononucleated cells (PBMCs) as described herein. After pulsing with at least one CSC, or fragments thereof, the immature dendritic cell may become a mature dendritic cell, optionally upon exposure to one or more maturation-inducing agent as described herein. The mature dendritic cell may have upregulated CD83, CD40 and CD86 expression. Advantageously, the mature dendritic cells of the present invention are capable of inducing specific immune responses of cytotoxic T lymphocytes against cancer stem cell antigens, such as colorectal cancer stem cell antigens.

The colorectal cancer stem cell may be an in vitro (test-tube derived) colorectal stem cell obtained by reverting at least one differentiated tumor cell to undifferentiated stem-cell state in vitro. The terms “in vitro colorectal stem cell,” “test-tube colorectal stem cell,” and “test-tube derived colorectal stem cell” are used interchangeably herein.

The terms “revert” or “reprogram” may refer to the conversion of a differentiated tumour cell back to a cell that is undifferentiated or non-fully differentiated, i.e. a cancer stem cell. For example, a differentiated colorectal tumour cell, such as a SW480 or HCT-9 cell may be “reverted” or “reprogrammed” into a colorectal cancer stem cell.

The term “differentiated colorectal tumour cell” may refer to a primary colorectal tumour cell (i.e. one that is obtained directly from a subject) that is fully differentiated (i.e. it does not have the ability to further differentiate). It may also refer to a colorectal tumour cell line that is fully differentiated. Exemplary differentiated colorectal tumour cells include human intestinal adenocarcinoma HCT-9 cells or human colon adenocarcinoma SW480 cells. A differentiated colorectal tumour cell may also include, but is not limited, to a human colon adenocarcinoma SW620 cell, a human colon adenocarcinoma CACO-2 cell, a human colon adenocarcinoma LoVO cell, a human colon adenocarcinoma COLO 205 cell, human colon carcinoma HT55 cell and a human colon carcinoma HT155 cell.

The term “undifferentiated” when used in reference to a cell, refers to a cell that is able to differentiate into one or more specialized cell types. An “undifferentiated” cell is therefore a cell that is not differentiated or a cell that is not fully differentiated. An “undifferentiated cell” may be a stem cell, an induced pluripotent stem cell, or a dedifferentiated cell. For example, an “undifferentiated” cell may be a colorectal tumour cell that has been reprogrammed to an undifferentiated colorectal stem cell state. An “undifferentiated” cell may be pluripotent, multipotent, oligopotent or unipotent.

A “colorectal cancer stem cell” may refer to a colorectal cancer cell that is undifferentiated, dedifferentiated, or not fully (partially) differentiated. In one example, the “colorectal cancer stem cell” is in an “undifferentiated colorectal stem cell state”. The “colorectal cancer stem cell” may therefore have the potential to differentiate into a variety of differentiated colorectal cancer cells. It may also have the ability to continue to proliferate and produce more “colorectal cancer stem cells”.

In one example, the colorectal cancer stem cell is an undifferentiated colorectal cancer stem cell that expresses transcription factors capable of reverting or reprogramming a differentiated colorectal tumour cell into an undifferentiated colorectal cancer stem cell state in vitro.

In one example, the undifferentiated colorectal cancer stem cell is obtained by reprogramming the differentiated colorectal tumour cell with transcription factors capable of reverting or reprogramming a differentiated colorectal tumour cell into an undifferentiated colorectal cancer stem cell.

In another example, the colorectal cancer stem cell is obtained by reverting or reprogramming the differentiated colorectal tumour cell with cell reprogramming factors or transcription factors. The cell reprogramming factors or transcription factors, may include, but are not limited to Oct4 and Sox2.

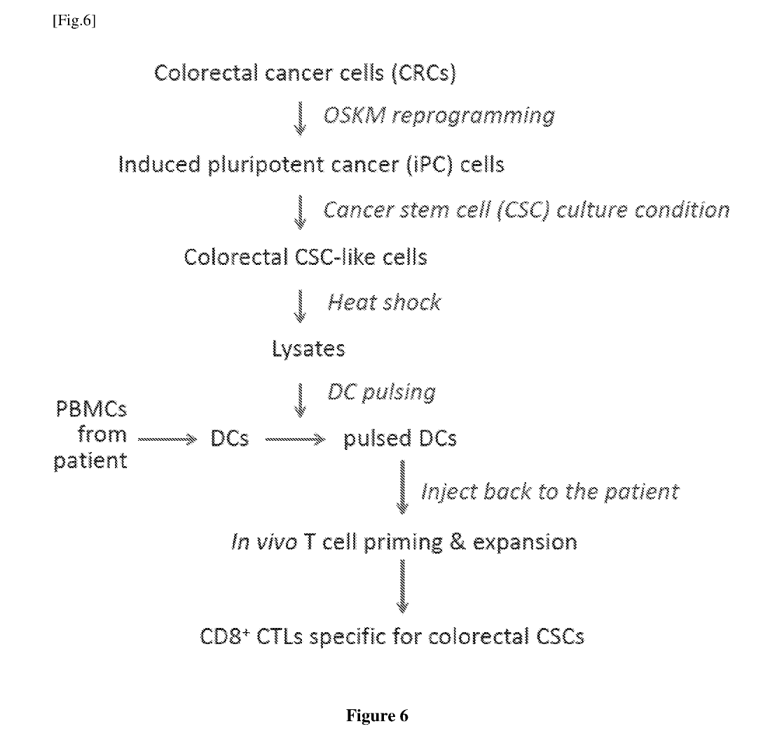

A “colorectal cancer stem cell” may express a range of colorectal stem-cell markers, or “tumor associated” antigens, such as Oct4 and Sox2. Some of these markers may be necessary to maintain the cancer stem-like properties of the cell. A “colorectal cancer stem cell” may be a colorectal CSC-like cell. A colorectal CSC-like cell may be obtained by OSKM reprogramming of a colorectal cancer cell (CRC) into an induced pluripotent cancer (iPC) cell, and culturing the induced pluripotent cancer (iPC) cell under cancer stem cell (CSC) culture condition to produce a colorectal CSC-like cell as shown in FIG. 6.

A colorectal cancer stem cell of the present disclosure may be prepared in vitro. In other words, the colorectal cancer stem cell may be prepared in a test tube, hence the terms “in vitro colorectal stem cell,” “test-tube colorectal stem cell,” and “test-tube derived colorectal stem cell”. Preparation of colorectal cancer stem cells in this way overcomes two major technical barriers in using cancer stem cells for DC cancer immunotherapy. These two barriers are: 1) cancer stem cells are rare in primary tumours, hence it is difficult to isolate and obtain enough cells for DC vaccination; and 2) cancer stem cells isolated from primary tumours will rapidly differentiate into the non-CSCs that represent the majority of cells in tumours in vitro, thus being unable to provide cancer stem cell antigens for DC vaccination. The present disclosure therefore enables the large-scale production of cancer stem cells for use in clinical cancer immunotherapy.

A special surprising advantage of the present disclosure is that test-tube cancer stem cells can provide sufficient quantities and a broad spectrum of tumour antigens, which can be grouped into three categories: 1) Pluripotency-associated antigens, including Oct4 and Sox2: the genes encoding pluripotency-associated antigens are site-specifically introduced into cancer cell genome for stable and high-level expression, which is rarely seen in cancer stem cells isolated from primary tumours; 2) Cancer stem cell-associated genes: test-tube cancer stem cells express many cancer stem cell-associated tumorigenic antigens that arise as a result of the pluripotency gene-mediated cell reprogramming; and 3) Test-tube cancer stem cells are derived from differentiated tumour cells, and therefore they also carry original somatic mutation, internal deletions, chromosome translocation in the parent cancer cells and many unidentified neoantigens associated with the parent cancer cells.

The use of test-tube cancer stem cells, which can be cultured as a stable cancer cell line, provides many technical advantages for vaccine preparation and application. In this way, immune protection may be provided against an antigen not recognized as a self-antigen by the immune system. Firstly, the use of an established cell line helps to circumvent a time-consuming and cost-intensive patient-individualized GMP production and eliminates the need for the continuous production of tailor-made individual vaccines. Secondly, the use of an established cell line simplifies the logistics, reduces the laboriousness of the vaccine production and delivery process, and increases its cost-effectiveness. Thirdly, the use of an established cell line allows for the highly standardized and large-scale production of allogeneic vaccines, so called “off-the-shelf” products suitable for all patients with a particular tumour type. Lastly, the use of a single batch of allo-vaccines for all vaccines, independent of HLA haplotype, eliminates variability in the quality and composition of the vaccines, facilitating reliable comparative analysis of clinical outcome.

In a second aspect, there is provided a composition for stimulating the immune system of a subject comprising at least one antigen presenting cell pulsed by contact to one or more antigens obtained from a colorectal cancer stem cell, wherein the colorectal cancer stem cell is obtained by reverting at least one differentiated colorectal tumour cell to have undifferentiated colorectal stem-cell state, wherein the reversion is obtained by reprogramming the differentiated colorectal tumour cell obtained from the subject with cell reprogramming factors or transcription factors including, but not limited to Oct4 and Sox2.

The term “stimulating” the immune system refers to activating the various components of the immune system, such as for example activation of cytotoxic T-cell lymphocytes, to respond to a particular threat. For example, the immune system may be stimulated to target and remove cancer cells. The immune system may be stimulated by one or more antigens or a vaccine to respond to such threats.

The antigen presenting cell and the colorectal tumour cell may both be obtained from the same or different subject. In other words, both antigen presenting cell and colorectal tumour cell may be autologous cells, or they may be allogeneic cells. In one example, both the antigen presenting cell and colorectal tumour cell are obtained from the same subject. Hence, both antigen presenting cell and colorectal tumour cell are autologous cells. In another example, the antigen presenting cell and colorectal tumour cell are obtained from different subjects. Hence, the antigen presenting cell and the colorectal tumour cell are allogeneic cells. These allogeneic cells are genetically dissimilar and may therefore be immunologically incompatible even though both subjects are of the same species.

The cell reprogramming factors or transcription factors may be delivered into the colorectal tumour cell using various methods known in the art, such as by use of a vector. The vector may comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product, such as Oct4 and Sox2. Selection of these and other common vector elements are conventional and many such sequences can be derived from commercially available vectors. The vector may be a plasmid vector, a viral vector, or any other suitable vehicle adapted for the insertion of foreign sequences and introduction into eukaryotic cells, such as colorectal cancer cells. Preferably, the vector is an expression vector capable of directing the transcription of the DNA sequence of the cell reprogramming factors or transcription factors. Viral expression vectors include, for example, baculovirus-, epstein-barr virus-, bovine papilloma virus-, adenovirus- and adeno-associated virus-based vectors. In one example, the delivery is facilitated by use of a baculoviral vector.

In one example, the cell reprogramming factors or transcription factors are delivered into the colorectal tumour cell using a baculoviral vector comprising zinc-finger nuclease-coding sequences and a fusion gene comprising the cell reprogramming factors or transcription factors. A skilled person would also appreciate that other vectors, such as a bacterial vector (i.e. a plasmid), or other viral vectors, such as an adenoviral vector as described above, may be used to deliver the cell reprogramming factors or transcription factors into the cell.

The undifferentiated state of a colorectal stem-cell that has been reprogrammed may be identified based on various characteristics that are unique to cancer stem cells. For example, the undifferentiated colorectal stem cell may be characterised by the loss of epithelial characteristics with reduction in E-cadherin expression. Alternatively, or additionally, the undifferentiated colorectal stem-cell state may also be characterised by the gain of mesenchymal properties with differential expression of, for example, vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and/or SOX4. Yet alternatively, or additionally, the undifferentiated colorectal stem-cell state may be characterised by the expression of cancer stem cell markers that include, but are not limited to, CD24, CS133, CD144, CD166, aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1).

In one example, there is provided a composition as defined herein, wherein the undifferentiated colorectal stem-cell state is characterised by at least one of the following: (a) loss of epithelial characteristics with reduction in E-cadherin expression, (b) gain of mesenchymal properties with differential expression of vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and SOX4; and (c) the expression of cancer stem cell markers selected from the group consisting of CD24, CS133, CD144, CD166, aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1).

In a third aspect, there is provided a composition for pulsing a dendritic cell such that the dendritic cell is capable of inducing specific immune response of cytotoxic T lymphocytes against an in vitro colorectal cancer stem cell, the composition comprising at least one in vitro colorectal cancer stem cell-like cell, or fragments thereof, which has been enriched from a re-programmed in vitro colorectal cancer cell.

The term “enrich,” or grammatical variants thereof, refers to a process of increasing the amount of one entity over others in a mixture. For example, in a mixture of cells, a particular cell type (such as an in vitro colorectal cancer stem cell-like cell) may be “enriched” such that it is present in a higher amount (i.e. higher number) over other cell types (such as colorectal cancer cells which do not have stem-cell like properties) in the mixture. The in vitro colorectal cancer stem cell-like cell, or fragments thereof, may be one as described above.

In a fourth aspect, there is provided a method of producing a stimulated immune system cell comprising: contacting a colorectal cancer stem cell, or fragments thereof, with at least one immune system cell, wherein the colorectal cancer stem cell is obtained by reverting at least one differentiated colorectal tumour cell to its undifferentiated stem-cell state.

The colorectal cancer stem cell may be an undifferentiated cancer stem cell that expresses transcription factors capable of reverting or reprogramming a differentiated colorectal tumour cell into an undifferentiated cancer stem cell state. The undifferentiated colorectal cancer stem cell may be obtained by reprogramming the differentiated colorectal tumour cell with reprogramming factors or transcription factors capable of reverting a differentiated colorectal tumour cell into an undifferentiated colorectal cancer stem cell as described above.

The reversion from differentiated colorectal tumour cell to its undifferentiated stem-cell state may be obtained by reprogramming the colorectal tumour cell with cell reprogramming factors or transcription factors selected from the group consisting of Oct4 and Sox2 as described above. The colorectal tumour cell and the immune cell may be autologous cells or allogeneic cells as described above. The immune system cell may be an antigen presenting cell, such as a dendritic cell. In one example, the dendritic cell is an immature dendritic cell. In one example, the immature dendritic cell, upon exposure to a reprogrammed colorectal tumor cell, and optionally upon exposure to a maturation inducing agent as described above, may become a mature dendritic cell that has upregulated CD83, CD40 and CD86 expression.

The colorectal stem cell used in the disclosed method may be an in vitro (or test-tube derived) colorectal stem cell as described above. The cancer stem cell may further be a heat-shocked cancer stem cell, or fragments thereof. The fragment may be a lysate of a heat-shocked cancer stem cell. Heat shock conditions that may be applied are as described above and in the Examples.

The method as defined herein may be an in vivo, ex vivo or in vitro method.

The cell reprogramming factors or transcription factors may be delivered into the tumour cell using a vector as described above and in the Examples. The vector may be a baculoviral vector comprising zinc-finger nuclease-coding sequences and a fusion gene comprising the cell reprogramming factors.

As described above, the undifferentiated stem-cell state of the colorectal cancer stem cell used in the method may be identified by various characteristics, such as at least one of the following: (a) loss of epithelial characteristics with reduction in E-cadherin expression, (b) gain of mesenchymal properties with differential expression of vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and SOX4; and/or (c) the expression of cancer stem cell markers selected from the group consisting of CD24, CS133, CD144, CD166, aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1).

In a fifth aspect, there is provided a vaccine comprising at least one immune cell stimulated with the method as defined herein. The immune cell may be an antigen presenting cell. The antigen presenting cell may be a dendritic cell. In one example, the immune cell and tumour cell are autologous cells obtained from a subject who is receiving the vaccine. In another example, the immune cell and the tumour cell are allogeneic cells obtained from a different subject who is receiving the vaccine.

The vaccine may comprise a pharmaceutically acceptable carrier. The vaccine may also comprise an adjuvant, which increases the immunological response of the subject to the vaccine. Suitable adjuvants include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

In a sixth aspect, there is provided a stimulated immune cell obtained from the method as defined herein. The stimulated immune cell may be an antigen presenting cell, such as a dendritic cell.

The composition, vaccine, or stimulated immune cell as described above may be used in therapy, for example in the treatment of cancer, such as colorectal cancer.

In a seventh aspect, there is provided a method of treating a colorectal cancer in a subject, comprising: immunising the subject with a composition as defined herein or a vaccine as defined herein or an immune cell stimulated by the methods as defined herein.

In an eighth aspect, there is provided a use of a composition of as defined herein or a vaccine as defined herein or an immune cell stimulated by the methods as defined herein, in the manufacture of a medicament for treating a colorectal cancer in a subject.

The term “subject” refers to a human or other mammal and includes any individual it is desired to examine or treat using the methods, compositions, vaccines, and/or stimulated immune cells of the invention. However, it will be understood that “subject” does not imply that symptoms are present. Suitable subjects that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer). The terms do not denote a particular age. Thus, both adult and newborn individuals are intended to be covered. The subject will preferably be a human, but may also be a domestic livestock, laboratory subject or pet animal. In one example, the subject is a cancer patient, such as a colorectal cancer patient. The patient may be one at any stage of the cancer, for example stage I, stage II, stage III or stage IV. The patient may be one who is suffering from cancer recurrence or relapse.

As used herein, the term “treat” or “treatment” includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

In a ninth aspect, there is provided a method of producing an anti-colorectal cancer vaccine for a subject comprising autologous dendritic cells, comprising the steps of: (a) extracting and purifying peripheral blood mononuclear cells (monocytes) obtained from a sample from the subject; (b) cultivating the monocytes under conditions effective to induce monocytes to dendritic cell differentiation; (c) contacting the cultivated immature dendritic cells of (b) with a cancer stem cell, or fragment thereof, obtained by reverting at least one differentiated colorectal tumour cell to its undifferentiated colorectal stem-cell state; and (d) cultivating the colorectal cancer stem-cell loaded dendritic cells of (c) with a dendritic cell maturation-inducing agent; and (e) harvesting the colorectal cancer stem-cell loaded mature dendritic cells as the anti-cancer vaccine.

The “peripheral blood mononuclear cell” may be any blood cell having a round nucleus. For example, a peripheral blood mononuclear cell may be a lymphocyte, a monocyte or a macrophage. Peripheral blood mononuclear cell may be extracted and purified from a blood sample taken from a subject via techniques that are known in the art, for example, via the Ficoll-Paque™ technique and/or via cell sorting techniques, such as magnetic-activated cell sorting (MACS). The monocytes obtained from these techniques may be cultured “under conditions effective to induce monocytes to dendritic cell differentiation”. For example, HLA-A2+ human peripheral blood mononuclear cells may be cultured with granulocyte-macrophage colony-stimulating factor (GM-CSF) and interleukin-4 (IL4) as described below, to obtain immature dendritic cells. Therefore in one example, the condition of (b) in the method of the ninth aspect comprises cultivating the monocytes in a culture medium comprising GM-CSF and IL-4.

A “dendritic cell maturation-inducing agent” as defined herein refers to an agent that is capable of inducing, or causing maturation of a dendritic cell from an immature state. Examples of dendritic cell maturation-inducing agents include, but are not limited to, lipopolysaccharide (LPS) and interferon-gamma as described below.

In one example, there is provided a composition comprising at least one isolated immune system cell primed by contact to at least one colorectal cancer stem cell, or fragments thereof as described above, wherein the colorectal cancer stem cell as described above is obtained by reverting at least one differentiated colorectal tumour cell to have undifferentiated colorectal stem-cell state as described above. In this example, the isolated immune system cell may be for example, a T cell, and the contact may be indirect contact such as via a dendritic cell that has been pulsed with the colorectal cancer stem cell, or fragments thereof as described above.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

As used in this application, the singular form “a,” “an,” and “the” include plural references unless the context clearly dictates otherwise. For example, the term “an agent” includes a plurality of agents, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” as used in relation to a numerical value means, for example, +50% or +30% of the numerical value, preferably +20%, more preferably +10%, more preferably still +5%, and most preferably +1%. Where necessary, the word “about” may be omitted from the definition of the invention.

### EXAMPLES

### Example 1 Materials and Methods

Zinc-finger nuclease technology was employed to insert a set of cell reprogramming factors named as the OSKM factors (Oct4, Sox2, Klf4, and cMyc) into CRC cell genome for induced reprogramming of these cancerous cells. Using a tumorsphere formation method, CSC-like cells were enriched from the reprogrammed CRC cells. These CSC-like cells consistently displayed the up-regulated expression of CD24 and many other colorectal CSC-related antigens. These CSC-like cells were then used for dendritic cell (DC) vaccination, which has been tested as an adjuvant treatment for CRC. After autologous naïve T cells were primed with DCs pulsed with cell lysates of OSKM-expressing CRCs, T cells were interrogated in IFN-gamma EliSpot assays by restimulation with T2 cells loaded with CSC antigen-related peptides. Significantly increased IFN-gamma positive spots confirmed that the pulsed DCs were capable of eliciting anti-CSC antigen responses in autologous T cells. Hence, OSKM-expressing CRC cell lines can be used as a readily accessible source to provide CSC-related antigens for DC vaccination, an approach that can possibly improve therapeutic outcomes for patients with CRC.

Plasmid and recombinant BV Vectors

pFastBac1 (Invitrogen, Carlsbad, Calif.), a donor plasmid that allows the gene of interest to be transferred into a baculovirus shuttle vector (bacmid) via transposition, was used as a plasmid backbone to construct recombination plasmids for baculovirus generation. Zinc-finger nuclease (ZFN)-coding sequences were subcloned into pFastBac1 donor plasmid as described previously (Phang et al., 2013; Tay et al., 2013). To construct pFB-ZFN, two DNA fragments encoding the right and left ZFNs, 993 bp each, were synthesized using GeneArt® Gene Synthesis service (Life Technologies, Carlsbad, Calif.) based on the amino acid sequences previously reported (Hockemeyer et al., 2009). The engineered ZFNs contain the right and left homologous arms pertaining to the AAVS1 locus fused with an obligate heterodimer form of the FokI endonuclease (Miller et al., 2007). The synthesized constructs were cloned into pMA (ampR) (Life Technologies). The two fragments were then amplified by PCR and subcloned into pFastBac1 using NotI/XbaI for the right ZFN and KpnI/HindIII the left ZFN respectively. The 1.1 kb human elongation factor 1α (EF1α) promoter was then amplified from pFB-EF1α-EGFP-hyg-lox (Ramachandra et al., 2011) and cloned into the above construct using BamHI/NotI to drive the expression of ZFNs. Finally, a 0.6 kb internal ribosome entry site (IRES) was amplified from pIRES (Clontech, Mountain View Calif.) and inserted between the right and left ZFN ORFs using XbaI/KpnI. AAVS1 begins 424 bp upstream of the 5′-end of exon 1 of the PPP1R12C gene and ends 3.35 kb downstream of the 3′-end. pFBZFN was used to target the region within intron 1 of the PPP1R12C gene.

The construction of the donor vectors pFB-OSKM and pFB-eGFP-OSKM bearing AAVS1 homologous arms was reported previously (Phang et al., 2013; Zhu et al., 2013). The OSKM expression cassette contains the EF1a promoter, a fusion gene (OSKM) composed of human Oct4, Klf4, Sox2, and C-myc genes joined with self-cleaving 2A sequence and IRES, and the woodchuck hepatitis virus post-transcriptional regulatory element (WPRE). To construct the donor plasmid pFB-OSKM with the reprogramming factors, an 810 bp left homologous arm and an 837 bp right homologous arm pertaining to the AAVS1 locus were amplified from pZDonor-AAVS1 (Sigma-Aldrich, St Louis, Mo.) and inserted using SnaBI/SalI for the left homology arm and NotI/BstBI for the right homology arm into pFB-PGK-Neo-EGFP-LoxP, a pFastBac1 vector previously constructed (Ramachandra et al., 2011), which contains heterospecific or both wild-type loxP sites. Upon removal of eGFP-coding sequences, a 63 bps adaptor sequence bearing EcoRI-AscI-SbfI restriction sites was introduced into a pFB-AAVS1 using EcoRI and SbfI. Then, the SV40 poly(A) signal was inserted through AscI and SbfI restriction sites.

Finally, a polycistronic cassette, containing the EF1a promoter, 4 iPSC transcription factor genes (human Oct4, Klf4, Sox2 and C-myc genes joined with self-cleaving 2A sequence and IRES as a fusion gene), and the woodchuck hepatitis virus post-transcriptional regulatory element was amplified from pHAGE-EF1a-STEMCCA (Millipore, Bedford, Mass.) and inserted into modified pFB-AAVS1 plasmid using EcoRI/AscI to construct pFB-OSKM. To construct pFB-eGFP-OSKM, a complete eGFP-coding sequence was introduced into the pFB-OKSM though a single EcoRI restriction enzyme and re-ligate. Calf Intestinal Alkaline Phosphatase (CIAP) was added to prevent self-ligation of the digested backbone vector. Primers used for vector construction are listed in Supplementary Table 1 below.

Recombinant BVs, including BV-ZFN, BV-OSKM, and BV-eGFP-OSKM, were generated using pFBZFN, pFB-OSKM, and pFB-eGFP-OSKM, respectively, and propagated in Sf9 insect cells according to the protocol of the Bac-to-Bac Baculovirus Expression System from Invitrogen. Recombinant DNA research in this study followed the National Institutes of Health guidelines.

### Generation of Human Induced Pluripotent Cancer Cells and Derivation of Colorectal CSCs

Human intestinal adenocarcinoma HCT-8 cells and human colon adenocarcinoma SW480 cells were originally provided by Dr. Lin Qinsong in National University of Singapore and cultured in Dulbecco's modified Eagle's medium (DMEM) (high glucose) medium with 10% fetal bovine serum (FBS, Invitrogen, Carlsbad, Calif.). Single-cell cloning was performed using limiting-dilution method. Randomly selected HCT-8 and SW480 subclones were used for genetic modification to introduce the OSKM genes into the AAVS1 locus. Specifically, 1×10⁴ cancer cells were seeded into one well of a 6-well plate in complete growth medium one day before baculoviral transduction. The cells were co-transduced with the BV-ZFN and BVeGFP-OSKM (or BV-OSKM) vectors at a multiplicity of infection (MOI) 100 plaque forming units (pfu) per cell each for 6 hours. Next day, the cells were transduced again under the same conditions described above. G418 selection at 400 μg/ml was started at day 3 post-transduction, and the medium was changed daily for 7 days. On day 10, the transduced cancer cells were dissociated and 1×10³ cells and re-plated onto one well in a fresh 6-well plate seeded with mitomycin C-inactivated mouse embryonic fibroblasts (MEF) in a human iPSC medium consisting of 80% DMEM/F12, 20% KnockOut Serum Replacer (Invitrogen), 2 mM L-glutamine, 0.1 mM β-Mercaptoethanol (Sigma-Aldrich), 0.1 mM nonessential amino acids (Invitrogen), 10 ng/ml bFGF (PeproTech, Rocky Hill, N.J.), and penicillin/streptomycin. The medium was replaced daily. On days 20-25, iPS cell-like colonies that were compact with defined borders were mechanically isolated and expanded on MEFs in the human iPS cell medium into iPC cell clones. These HCT-8 and SW480-derived iPC cell colonies were manually passaged every 7 days. The cells were maintained in the undifferentiated state by scraping off differentiated cells with a pipette or by mechanical passage of individual colonies of undifferentiated cells.

Prior to differentiation of HCT-8 and SW480-derived iPC cells into colorectal CSCs, iPC cell colonies were expanded under feeder-free conditions on Matrigel-coated plates (BDscience, Franklin Lakes, N.J.) with mTesR culture medium (STEMCELL Technology, Vancouver, BC, Canada) under a standard cell culture condition (37° C., 5% CO₂) in a humidified incubator. The expanded cells were dissociated to single cells using Accumax (Millipore, Bedford, Mass.) and plated onto a 0.1% gelatin-coated six-well cell culture plates (Nalge Nunc International, Rochester, N.Y.) at a density of 1×10⁴ per well. The cells were cultured in an CSC medium composed of DMEM/F12 (1:1 mixture) medium (Invitrogen) supplemented with 1% FBS, 2% B27 (Invitrogen), 2 mM L-glutamine, 50 unit/ml penicillin, 50 μg/ml streptomycin and 20 ng/ml human epidermal growth factor (EGF) (Sigma-Aldrich). After 1 month of passaging, a homogeneous cell population was achieved for CSC characterization. For passaging, the cells were washed with 1× Dulbecco's phosphate buffered saline (Invitrogen), treated with 0.25% (w/v) trypsin-0.53 mM EDTA, and subcultured at a split ratio of 1:10 on uncoated T25 culture flask. These cells could be cryopreserved in the CSC complete growth medium containing 10% DMSO and remained viable after thawing from liquid nitrogen storage.

### Genomic DNA Extraction and Genotyping

Genomic DNA of cells was isolated using a DNeasy blood & tissue kit (Qiagen, Hilden, Germany) PCR genotyping was used to verify the site-specific integration of the OSKM expression cassette at the AAVS1 site driven by the ZFN-mediated homologues recombination. An optimized reaction buffer consisted of 21.5 μL Platinum® Taq DNA polymerase high fidelity master mix (Invitrogen), 0.5 μL for 10 μM of each forward and reverse primer, 1.5 μL DMSO and 1 μL of 200 ng/μL DNA template. PCR amplifications of genomic DNA were performed using the following parameters: an initial denaturation step at 94° C. for 5 min followed by 35 cycles at 94° C. for 25 s, 65° C. for 45 s and 72° C. for 150 s with a final extension step at 72° C. for 10 min. Amplified products were analyzed on a 1% agarose gel. Primers used for PCR amplification are listed in Supplementary Table 1 below.

For Southern blot analysis, genomic DNA (10 μg) was digested overnight with EcoRI. The digested DNA was loaded on a 1% agarose gel and electrophoresis was performed for 10 hours at 25V. Using the iBlot® Dry Blotting System (Invitrogen), the DNA was then transferred to the iBlot® DNA Transfer Stack (Invitrogen) containing a positively charged nylon membrane. The membrane was incubated in 1.5 M NaCl/0.5 M NaOH denaturing solution for 10 min immediately after transfer and air-dried. After UV crosslinking at 130 mJ/cm², the membrane was hybridized overnight with DIG Easy Hyb (Roche, Indianapolis, Ind.). DIG-labeled probes targeting the WPRE region of the OKSM expression cassette were synthesized using the PCR DIG Probe Synthesis Kit (Roche). Following hybridization the membrane was stringent washed, blocked, and then incubated with an anti-digoxigenin-AP conjugate (DIG DNA Labeling and Detection Kit, Roche) that was detected by CDP-Star, ready-to-use (Roche). The membrane was first washed twice with 2×SSC/0.1% SDS at 40° C. and then twice with 0.1×SSC/0.1% SDS at 65° C. Blocking and washing were performed using the DIG Wash and Block Buffer Set (Roche). The membrane bearing DNA was exposed to Chemiluminescent Image Analyzer (ImageQuant LAS 4000 mini, GE Healthcare Biosciences, Pittsburgh, Pa.) for 15 min. Primers used for synthesizing DIG-labeled probes are listed in Supplementary Table 1 below.

### Quantitative Real-Time PCR

Total RNA was extracted and reversely transcribed using SuperScript III First-Strand Synthesis System (Invitrogen). For RT-PCR analysis, PCR amplifications were performed using the following parameters: an initial denaturation step at 94° C. for 5 min followed by 25-30 cycles at 94° C. for 15 s, 60° C. for 45 s and 72° C. for 30s with a final extension step at 72° C. for 5 min. Amplified products were analysed on a 2.5% agarose gel. PCR primer sets for endogenous, exogenous and total OSKM (human Oct4, Sox2, Klf4 and c-Myc genes) analysis are listed in Supplementary Table 1 below.

For quantitative real-time PCR (qPCR) analysis, the RT Real time SYBR Green PCR Master mix was used to amplify the synthesized cDNA. The housekeeping gene glyceraldehyde-3-phosphate dehydrogenase (GAPDH) was used for internal normalization. The 2× QuantiTect SYBR Green PCR master mix was used to amplify the cDNA in a two-step RT-PCR. The normalization of expression levels of genes of interest was done by dividing their relative expression level by the relative expression level of GAPDH. Relative gene expression level was obtained by triplicate experiments for each gene. Relative quantification of gene expression was evaluated using the ΔΔCt method. The fold change in the relative gene expression was determined by calculating 2^{-ΔΔCt}. qPCR analysis was performed to quantify the expression of colorectal CSC markers, epithelial-mesenchymal transition (EMT) markers, commonly used colorectal cancer diagnosis and prognosis markers, and a set of the most significantly colorectal cancer associated loci identified in genome-wide association studies (GWASs).

### Immunofluorescence Staining, Western Blot Analysis, and Flow Cytometry Analysis

iPC cell colonies were seeded on a Matrigel-coated, 24-well chamber slide and fixed in 4% paraformaldehyde for 30 min at room temperature. To induce the permeability of cells, 0.1% triton was added and incubated for 10 min. After washing with PBS, the cells were incubated in a blocking solution (5% BSA) for an hour. Primary antibodies used are those against NANOG (1:100, R&D systems, Minneapolis, Minn.), TRA1-60 (1:100, Millipore), and SSEA-4 (1:200, Santa Cruz). Goat anti-rabbit IgG-FITC or mouse anti-rabbit IgG-Rodamine (Santa Cruz Biotechnology) was used as the secondary antibody. After antibody incubation, the samples were stained by DAPI (1:1000, Chemicon) for nucleus staining. The images were then photographed by a fluorescence microscope.

For Western blot analysis, cells were lyzed with RIPA buffer containing a protease inhibitor cocktail (Nacalai Tesque). Protein concentrations of the lyzates were measured with a protein assay dye reagent (BioRad) using the Xmark microplate spectrometer (BioRad). Protein lysates were loaded for sodium dodecyl sulfate polyacrylamide gel electrophoresis (SDS-PAGE), and transferred to nitrocellulose membranes (BioRad). The membranes were blocked for 1 hour at room temperature and then incubated with mouse anti-CD24 (Beckman Coulter), rabbit anti-Sox2 (Abcam) and rabbit anti-Oct4 (Abcam) antibodies at 4° C. overnight and followed with HRP conjugated secondary antibodies Immunoreactive bands were visualized using Pierce ECL Western blotting substrate (Thermo Scientific). β-actin antibody was used to confirm equal loading.

For flow cytometry analysis, the following monoclonal antibodies: phycoerythrin (PE)- or allophycocyanin (APC)-labeled anti-CD133 (clone AC133/1; Miltenyi Biotec), APC-labeled anti-CD24 (clone 32D12; Miltenyi Biotec), APC-labeled anti-CD44 (clone DB105; Miltenyi Biotec), and PE-labeled anti-CD166 (clone 3A6; BD Pharmingen) were used for identification and enumeration of CSCs. Ten μL of each antibody conjugates was added up to 10⁷ nucleated cells per 100 μL of MACS buffer (PBS, pH7.2, 0.5% BSA, and 2 mM EDTA) for labeling of cells and subsequent analysis by flow cytometry. To correct for spectral overlap with the fluorophores FITC and PE, color compensation is required for flow cytometry analysis of eGFP positive cells.

### Tumor Sphere Formation Assays, Colony Formation Assay, Soft Agar Colony Formation Assay, Wound Healing Assay, and Cell Migration/Invasion Assay

Tumor sphere formation assays were performed as described previously with some modifications (Lo et al, 2012). Cells were grown in serum-free, non-adherent conditions in a 96-well ultra-low attachment plates (Corning, Corning, N.Y.) in the CSC medium. Two hundred cells were seeded into each well in a 96-well plate and 20 wells (4,000 cells totally) were tested for each clone. After one-week culturing, the number of tumor spheres were counted under a phase-contrast microscope using the 40× magnification lens. The tumor spheres should have a solid, round structure, with a size varying from 50 to 250 micrometers.

For colony formation assay, cells (400 cells/2 mL) in RPMI-1640 media plus 10% FBS were dispensed into 6-well culture plate and cultured for 3 weeks. The numbers of colonies formed were visualized with Gibco® KaryoMAX® Giemsa Stain Solution (Invitrogen) staining and were counted under phase contrast microscope.

For soft agar colony formation assay, cells were plated for a density of 1E4 per well in triplicate in 6-well plates with 0.6% base agar and 0.4% top agar and incubated in 37° C., 5% CO₂ for one month. The colonies were stained with MTT for 2 hours, photographed, and quantified with Image J.

For wound healing assay, cells were seeded in triplicate in 6-well plates and scratched using 200-ul tip after forming a confluent monolayer. At 0 and 48 hours after incubation, images were captured under the phase contrast microscope and the closure of scratch was analyzed.

For cell migration and invasion assays, cells were suspended in serum-free DMEM and seeded to the top chamber of 8-μm pore size transwell chambers (BD Bioscience) at a density of 5×10⁴ per well. The chambers were coated with Geltrex (Life Technologies) for invasion assays while the chambers without costing were used for migration assays. Cells were pre-labeled with Calcein-AM (5 μg/ml) (Life Technologies) by incubation at 37° C. for 30 minutes and seeded to the top chamber. The bottom chamber was prepared with 15% FBS as a chemoattractant. After incubation for 24 hours at 37° C., the cells that migrated and invaded through the membrane and stuck to the lower surface of the membrane were fixed with a fixative/staining solution (0.1% crystal violet, 1% formalin and 20% ethanol) for visualization and counted under a microscopy. Cell invasion rate was calculated by the number of cells invading through Geltrex divided by the number of cells migrating through uncoated insert membrane.

### Teratoma Formation Assay

To test the teratoma formation ability of iPC cells, 1×10⁶ cells were dissociated using Accutase (Millipore) and mixed with 0.5×10⁶ mitomycin-treated HFF in PBS to a total volume of 50 μl. Prior to transplantation, 50 μl undiluted cold Matrigel (Becton Dickinson, Franklin Lakes, N.J.) was added to the prepared cells. The cells were injected into the rear legs of 5-week-old NOD/SCID IL2Rg (null) (NSG) mice. Two months after injection the resulting teratomas were removed and fixed in 4% paraformaldehyde, embedded in paraffin, cut in 5-μm sections, and stained with haematoxylin and eosin. All handling and care of animals was performed according to the guidelines for the Care and Use of Animals for Scientific Purposes issued by the National Advisory Committee for Laboratory Animal Research, Singapore.

### Example 2

### Generation of Colorectal Cancer iPC Cells by Stable Expression of the OSKM Genes

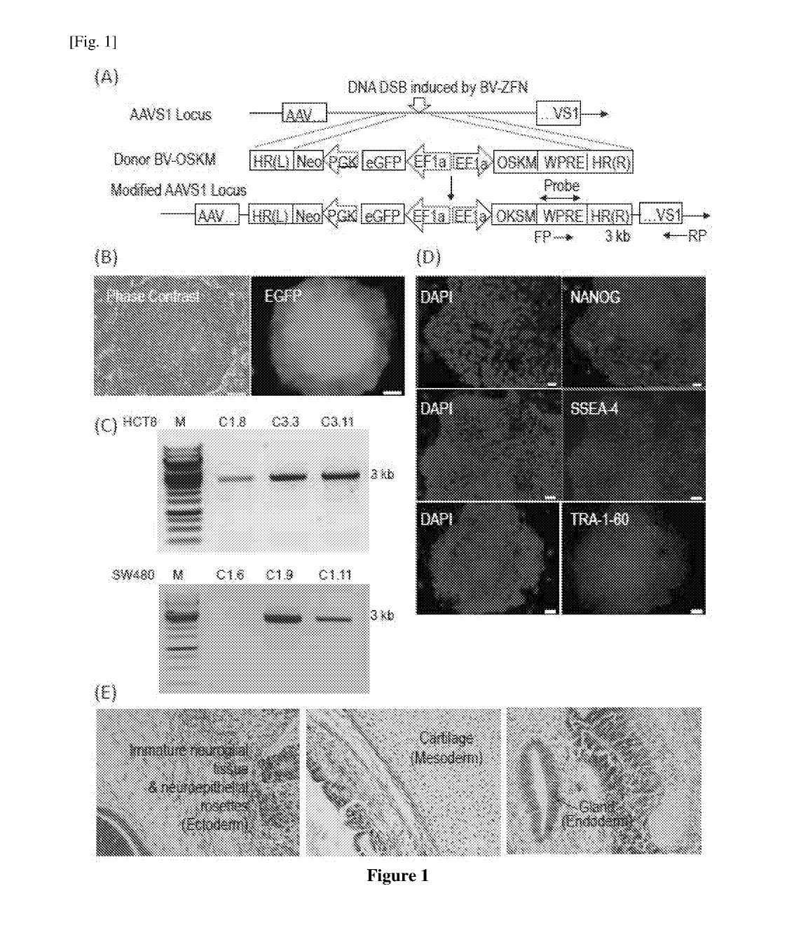

A baculoviral transduction-based engineered zinc-finger nuclease (ZFN) technology was recently developed for site-specific integration of the OSKM factor genes (Phang et al., 2013). The technology involves the use of two non-integrative baculoviral vectors, one expressing ZFNs (BV-ZFN) and another as a donor vector encoding the OSKM transcription factor genes (BV-OSKM). BV-OSKM carries an expression cassette containing human Oct4, Klf4, Sox2, and c-Myc genes joined with self-cleaving 2A sequence and IRES as a fusion gene and driven by the EF1a promoter. The expression cassette is flanked on both sides by sequences homologous to the AAVS1 locus. After co-transduction with BV-ZFN and BV-OSKM (FIG. 1A), the expression cassette can be effectively introduced into the AAVS1 locus in human chromosome 19, a site with an open chromatin structure flanked by insulator elements that shield an integrated transgene from gene silencing, thus facilitating robust and persistent transgene expression in the modified cells. Cancer cell re-programming in single-cell-derived subclones of human CRC HCT-8 and SW480 cells was tested with this technology.

After transferring the transduced cells to the feeder layer of mitomycin C-inactivated mouse embryonic fibroblasts (MEF) on day 15 post-transduction and culturing them in a human iPS cell medium, formation of early colonies could be observed around day 20. The colonies displayed compact cell morphology with sharp border (FIG. 1B) and were positive for AP staining. PCR genotyping confirmed the AAVS1 site-integration of the OSKM cassette in 5 out of 6 examined samples (FIG. 1C), which was further confirmed by Southern blot analysis. The generated colonies expressed classic embryonic stem markers as evidenced by immunostaining (FIG. 1D), RT-PCR analysis, and flow cytometric analysis. To examine their differentiation potential, the cells collected from the colonies were injected into the hind legs of NSG mice to form teratomas. The differentiation profile of the formed teratomas was assessed by histological examination and demonstrated the presence of cells from all three embryonic germ layers in a teratoma (FIG. 1E). These findings confirm that CRC cells can be reprogrammed into iPC cells and some of the reprogrammed cells display pluripotency.

### Example 3

### Ectopic Expression of OSKM Confers CSC-Like Properties to Human CRC Cells

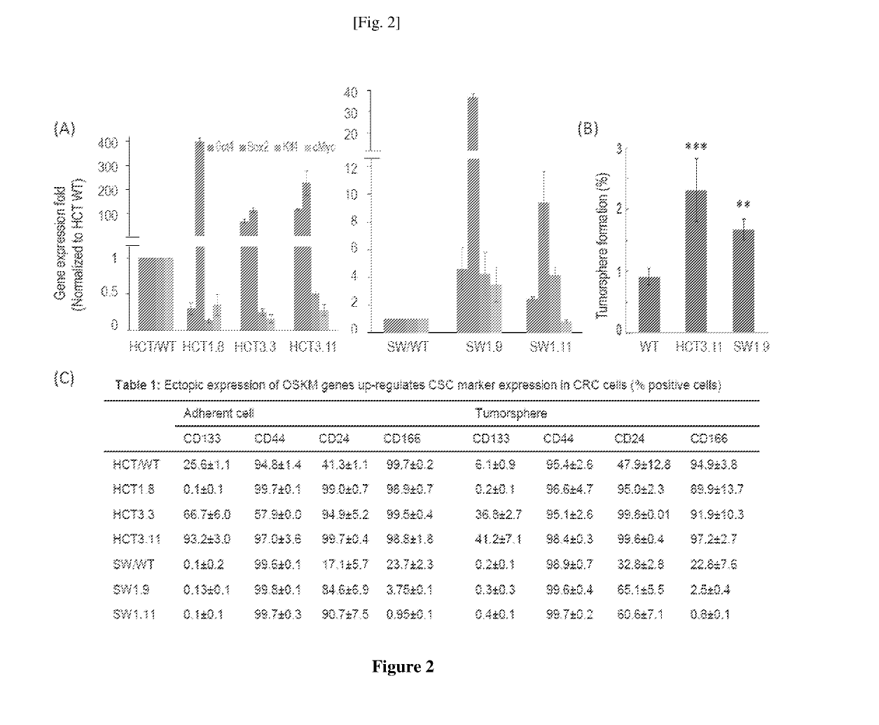

HCT8- and SW480-derived iPC cells were expanded in a CSC medium composed of 1:1 mixture of DMEM/F12 supplemented with 1% FBS and 20 ng/ml human epidermal growth factor (EGF). Using a RTqPCR method, the relative expression levels of OSKM genes in the selected clones were determined (FIG. 2A). Compared to wild-type (WT) cells, Sox2 over-expression, from 8- to 400-fold, was observed in all examined HCT8 and SW480 clones. Up-regulation of Oct4 was observed in 4 out of 5 examined clones, ranging from 2- to 100-fold. Western blot analysis confirmed the up-regulated expression of Oct4 and Sox2 proteins in these clones. Up-regulation of c-Myc and Klf4 was not so obvious, possibly because of high-level expression of the endogenous genes in the HCT8 and SW480 CRC subclones.

The roles of the ectopic expression of the OSKM gene in phenotypic features of CSCs were sought to be clarified. The ability to form tumorspheres is a characteristic of CSCs. When HCT3.11 and SW1.9 clones were dissociated to single cell suspensions and cultured in ultra low-attachment plates, significantly higher numbers of tumorspheres formed on day 14 were detected as compared with WT HCT8 and SW480 subclones (FIG. 2B). Expression of 4 well-studied CSC surface markers commonly used for CRCs, prominin 1 (CD133), CD44 antigen (CD44), small cell lung carcinoma cluster 4 antigen (CD24), and activated leucocyte cell adhesion molecule (CD166), was analyzed by flow cytometry in several of OSKMexpressing HCT8 and SW480 clones. CD24 up-regulation was consistently observed in all examined clones, even in a clone (HCT1.8) that displays Sox2 up-regulation only (FIG. 2C). A critical experiment for determining the CSC phenotype is to examine the in vivo tumorigenicity of testing cells by injecting serial dilutions into immunodeficient mice. An in vivo tumorigenicity test was performed by subcutaneous injection of 10,000 tumor cells into NOD SCID mice and tumor formation in 6 out of 10 mice after HCT3.11 inoculation was detected, while there was no tumor formation at all after inoculation of HCT/WT cells in 10 mice. In vitro assays performed with HCT3.11 and SW1.9 clones also confirmed increased colony formation, cell invasion, and cell motility. Thus, overexpression of Oct4 and Sox2 was sufficient to induce CSC properties in CRC cells.

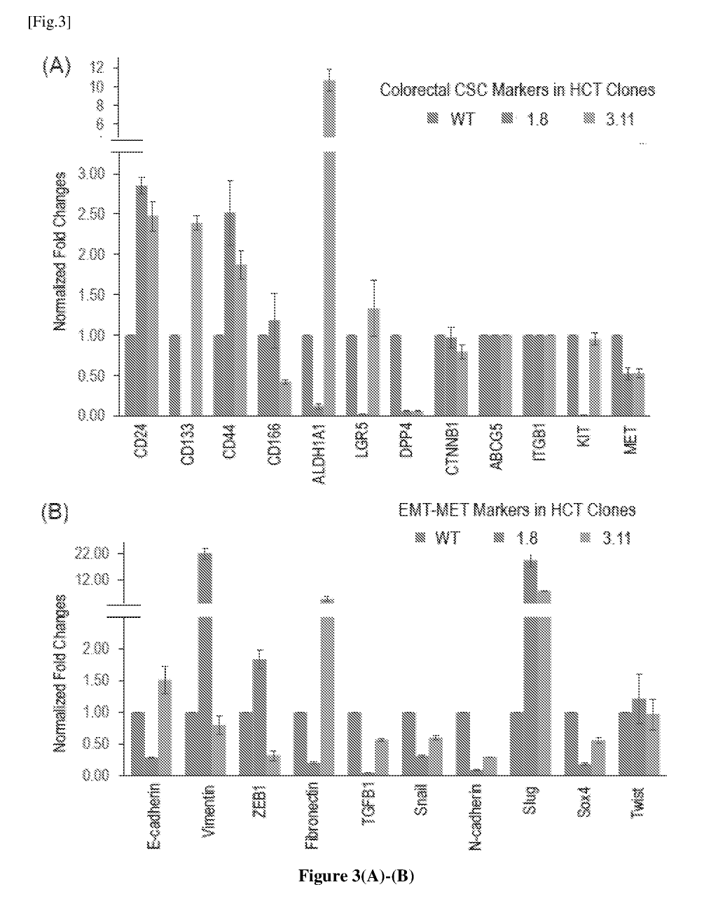

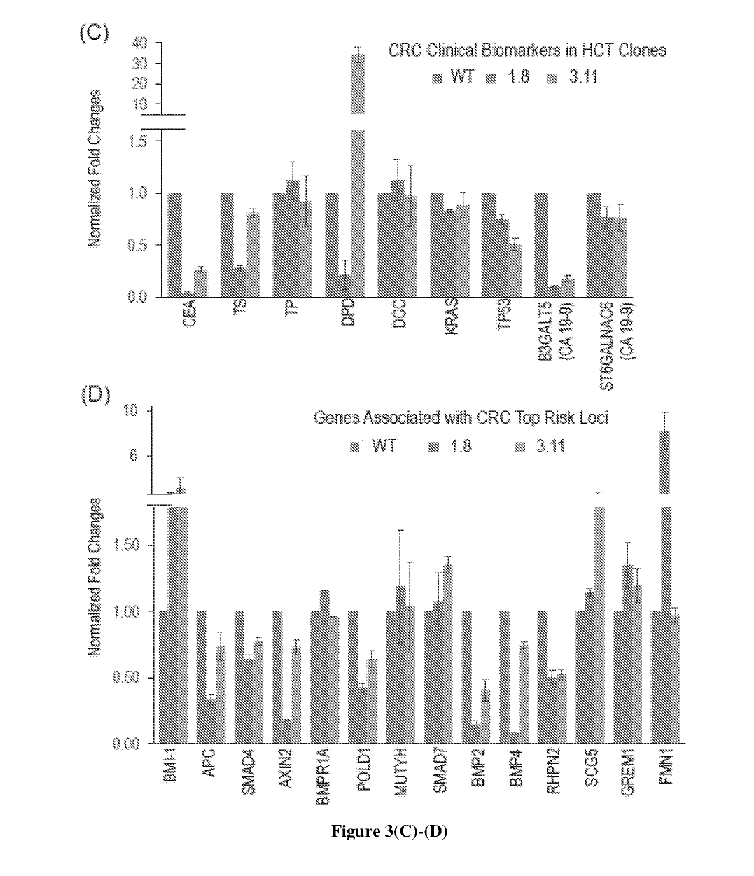

To further characterize the derived OSKM-expressing cells, three HCT clones, a single cell clone without OSKM modification (HCT/WT), HCT3.11 in which both Oct4 and Sox2 are up-regulated, and HCT1.8 with Sox2 up-regulation only, were subject to in depth assessment of marker gene expression with real-time RT-PCR analysis. In addition to CD24, CD133, CD44 and CD166, other tested colorectal CSC markers include aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1). Notably, a 10-fold up-regulation of ALDH1A1 gene expression in HCT3.11 was detected (FIG. 3A). Tumor cells expressing high levels of ALDH1A1 have been found to display the properties attributed to CSCs in leukemia and several types of solid tumors. Since CSC-like cells can be generated by aberrant activation of epithelial-to-mesenchymal transition (EMT), quantitative RT-PCR analysis was performed to determine the expression of EMT markers. The loss of epithelial characteristics will be examined by E-cadherin expression, whereas the gain of mesenchymal properties will be determined by vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and SOX4 expressions. Vimentin, ZEB1 and slug were found to be up-regulated in HCT1.8 and fibronectin and slug were re-regulated HCT3.11 (FIG. 3B). Real-time RT-PCR analysis was also performed to examine gene expression levels of CRC markers commonly used in clinical settings for diagnosis and prognosis, including carcinoembryonic antigen (CEA), CA 19-9 (B3GALT5 and ST6GALNAC6), thymidylate synthase (TS), thymidine phosphorylase (TP), dihydropyrimidine dehydrogenase (DPD), GTPase KRas (KRAS) and tumor suppressor p53 (TP53). DPD was significantly up-regulated, up to 30-fold, in HCT3.11 (FIG. 3C). Over-expression of DPD in tumor tissues is known to be associated with insensitivity to chemotherapy. The expression of 14 genes associated with the most significantly CRC-associated loci identified in GWASs was further quantified. The up-regulation of the polycomb complex protein (BMI-1) gene, the mothers against decapentaplegic homolog 7 (SMAD7) gene, and the neuroendocrine protein 7B2 (SCG5) gene in HCT3.11 and the upregulation of BMI1 and formin-1 (FMN1) genes in HCT1.8 were detected (FIG. 3D). The effects of OSKM-expression on various pathological markers associated CRCs indicate the important roles of these transcription factors in the tumorigenesis and development of CRC.

### Example 4

### Human Dendritic Cells Loaded with Lysates of OSKM-Expressing CRCs Elicit Colorectal CSC Antigen-Specific T-Cell Response after In Vitro Priming Autologous T Cells

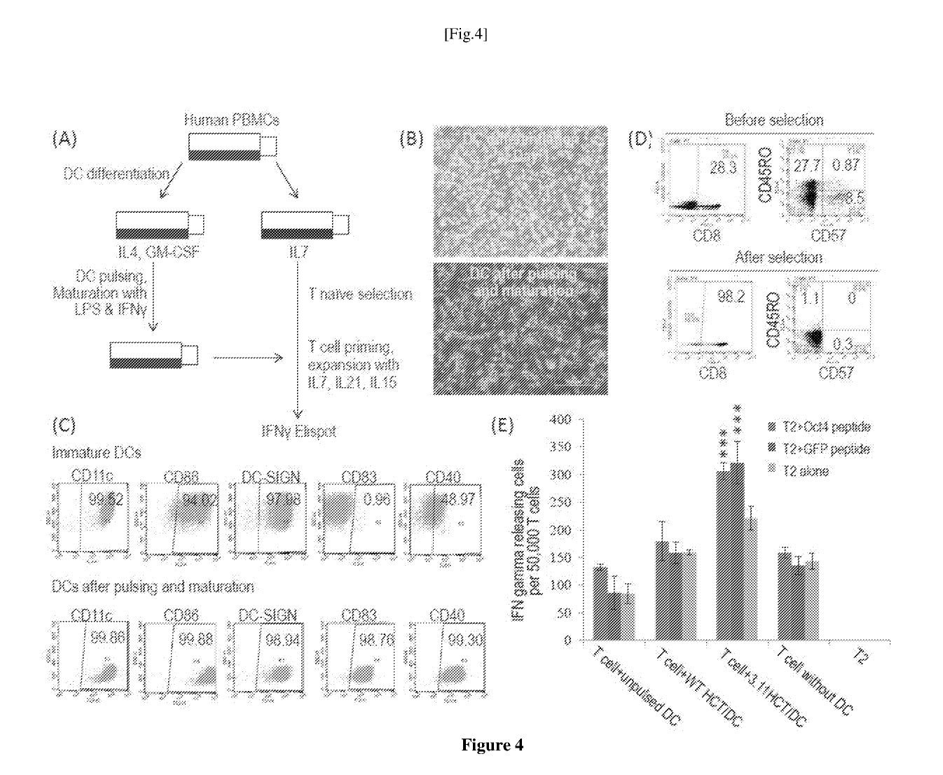

Given the increased CSC-like properties in OSKM-expressing CRC cells, DCs pulsed with these cells were assessed to determine whether they could be used to prime human T cells reactive against the Oct4 antigens (FIG. 4A). Immature DCs were generated from HLA-A2+ human peripheral blood mononucleated cells (PBMCs) with granulocyte-macrophage colony-stimulating factor (GM-CSF) and interleukin-4 (IL-4) (FIG. 4B). These DCs were positive for DC markers such as CD11c, CD86 and DC-SIGN, but displayed a low level expression of T cell co-stimulatory molecule CD40 and almost no expression of CD83, a molecule important for stimulating T cells (FIG. 4C). To initiate immune responses, DCs should maturate from antigen processing cells to antigen-presenting cells (APCs). Upon maturation with lipopolysaccharide (LPS) and interferon-gamma (INF-gamma), PBMC-derived DCs up-regulated the expression of CD83 and CD40 and CD86, from 0.96% and 48.97% in immature DCs to 98.76% and 99.30% in mature DCs for CD83 and CD40, respectively (FIGS. 4B, C). Naïve T cells were selected also from PBMCs using MACS, which increased CD8+ cells from 28% to 98% and significantly reduced CD45RO+ and CD57+ memory T cells (FIG. 4D). These naïve T cells were then primed with HCT3.11 tumor cell lysate-pulsed DCs to generate CTLs. The frequency of specific T cells recognizing individual antigens after immunizations are usually very low. ELISPOT technology, a method that detect individual cytokine secreting cells, is highly sensitive in measuring T cell immunity, possibly to detect antigen-specific T cells in frequency range of 1:10,000 to 1:1,000,000. IFN-gamma ELISPOT assays were thus performed to analyze antigen-specific T cell responses against Oct4. Significantly increased T cell reactivity after restimulation with T2 cells loaded with Oct4 and GFP (positive control) peptides (FIG. 4E) demonstrated that human DCs loaded with lysates of OSKM-expressing CRCs is capable of eliciting anti-CSC antigen responses in autologous T cells.

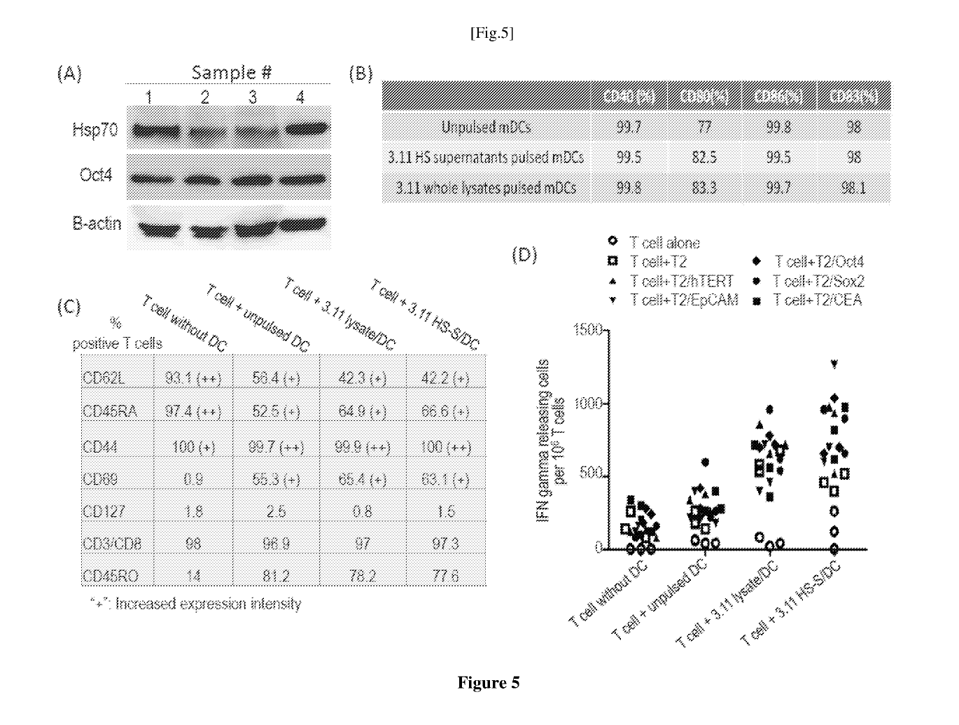

Since dying tumor cells provide both a source of tumor antigens and endogenous danger signals that are capable of triggering antigen presenting cell activation, heat stress has been used to induce tumor cell death before using their lysates for DC pulsing. Heat stress strongly increases the levels of heat shock proteins (HSPs), a superfamily of distinct proteins that are expressed constitutively at low levels and significantly induced under conditions of cellular stress. In the immune system, these proteins can both induce the maturation of DCs and provide chaperoned polypeptides for specific triggering of the acquired immune response. To test whether stressed dying tumor cells can be used as a superior antigen source for DC pulsing, OSKM-expressing HCT3.11 cells were heat-treated at 42° C. for 60 min. After culturing at 37° C. for 2 hours for cell recovery, the heat-treated cells were frozen and thawed for 6 cycles and the cell supernatants were collected for DC pulsing. The upregulation of the Hsp70 protein was observed without affecting Oct4 expression in heat-treated cells (FIG. 5A). After pulsing DCs using the supernatants collected from the heat-shocked cells, DC surface marker expression was examined and no differences were observed between tumor supernatant-pulsed DCs and DCs maturated without pulsing (FIG. 5B). These DCs were then used for T cell priming After two consecutive weeks' co-culturing with the DCs, CD8⁺ T cells were expanded 9 times, while for T cells primed using unpulsed DCs 6-fold expansion was observed. T cell marker expression was further examined after the cells were primed with DCs. The T cells primed with DCs pulsed with the supernatants collected from the heat-shocked cells had become effector T cells and some of them were detected to be memory T cells (FIG. 5C). Using IFN-gamma ELISPOT assays, increased antigen-specific T cell responses against Oct4, Sox2 and several other CSC- and CRC-associated antigens were detected after T cells were primed with DCs pulsed with the supernatants collected from the heat-shocked cells as compared with T cells primed with whole lysates of HCT3.11 without heat shock (FIG. 5D). These findings suggest that uptake of apoptotic bodies of heat-shocked tumor cells can generate mature DCs that are more adept at eliciting CSC antigen specific CTLs.

### Example 5

A number of clinical trials have proved the safety and efficacy of the autologous dendritic cell (DC)-based cellular immunotherapy. Given the clinical observations that up-regulation of Oct4, Sox2, CD24, and many other CSC markers is associated with poor prognosis in multiple human cancer types, a new strategy for DC vaccination has been developed here against CSCs (FIG. 6), where it has been demonstrated that cell lysates of the OSKM-expressing CRC cells could be used as an accessible source of antigens associated with undifferentiated CSCs for DC pulsing to induce specific immune responses against colorectal CSCs. As shown in FIG. 6, colorectal cancer cells (CRCs) are first subjected to “OSKM reprogramming” (i.e. reprogramming using an OSKM expression cassette comprising Oct4, Sox2, Klf4 and c-Myc genes) into induced pluripotent cancer (iPC) cells. The iPC cells are cultured under cancer stem cell (CSC) culture conditions as described herein to obtain colorectal CSC-like cells. The CSC-like cells are then subjected to heat shock treatment and lysed to produce a lysate containing the desired tumour antigens. The heat shocked lysate is used to pulse immature DCs, prepared from peripheral blood mononuclear cells (PBMCs) that have been obtained from a patient using the methods as described herein. The pulsed DCs are then injected back into the patient to induce in vivo T cell priming and expansion. This generates CD8+ CTLS specific for the colorectal CSCs.

### Expression of Oct4 and Sox2 Genes in Cancer Stem Cells

A significant development in the field of stem cell biology is the generation of induced pluripotent stem (iPS) cells through reprogramming of differentiated somatic cells with the OSKM transcription factors. Oct4 and Sox2, together with Nanog, are the three genes encoding the core elements of the regulatory circuitry responsible for maintaining the pluripotency of embryonic stem (ES) cells. Klf4 plays important roles in both carcinogenesis and normal development, especially in the transcription regulatory network important for self-renewal and pluripotency in ES cells and iPS cells. Myc is a well-studied classical oncogene and one of the most highly amplified oncogenes in many different human cancers.

High similarities between iPS cell derivation through reprogramming and oncogenic transformation have been noticed: both can be induced by a series of genetic and epigenetic modifications and utilize similar transcriptional networks. During the process of changing from somatic differentiated cells to iPS cells, the cells acquire unlimited proliferation properties and self-renewing activities, the two characteristics that are most important for cancer cells. Indeed, studies have shown that the iPS genes are expressed in various types of tumors and involved in their development, supporting the notion that cell reprogramming and tumor transformation utilize common mechanisms. Ectopic expression of Oct4 can dose-dependently increase the malignant potential of embryonic stem cells and is sufficient to induce tumors in mice. Sox2 has been identified as an amplified lineage-survival oncogene in lung and esophageal squamous cell carcinomas. An increased expression of pluripotency-related factors is frequently detectable in poorly differentiated solid tumor, suggesting that those malignant cells have gained undifferentiated, stem cell properties. Clinically, the increased expression of Oct4 and Sox2 in subtypes of cancers is associated with aggressive disease courses, such as undifferentiated histology, resistance to therapy, metastases, relapse, and shorter patient survival rates. Because of the correlations, the two proteins have been proposed as novel predictors of distant recurrence and poor prognosis in cancer patients.

Recently, direct evidence linking the Oct4 and Sox2 genes to CSCs has emerged. The two proteins are found to be enriched in CSCs in several cancers as compared to relatively differentiated cancer cells with same derivation. In lung cancer-derived CD133-positive cells, Oct-4 expression is needed to maintain cancer stem-like properties of the cells. In glioblastoma, knockdown of Sox2 leads to the inhibition of proliferation and loss of tumorigenicity of glioma CSCs. In ostersarcoma, a subpopulation of tumour cells have been identified that are capable of activating an exogenous Oct4 reporter and are more tumourigenic than their counterparts in reconstituting heterogenous tumours with both Oct4-positive and Oct4-negative cells. In breast cancer, ectopic expression of Oct4 in normal breast cells lead to the generation of cells with tumor-initiating and colonization abilities and development of high-grade, poorly differentiated breast carcinomas in nude mice. In epithelial ovarian cancer, CSCs isolated from ovarian primary tumors have an enhanced expression of Oct4. In CRC, the ectopic expression of the iPS genes promotes sphere-formation, proliferation, colony formation and migration of human CRC cells, and the signature for iPS gene expression in CRCs can be used to predict the survival of cancer patients. These findings suggest that the iPS genes might be the master regulators of CSC induction and the key factors in the maintenance of CSC identity. Being the proteins that are overexpressed on CSCs, or highly active in these tumorigenic cells, but have limited expression in normal tissue, the Oct4 and Sox2 products are potential targets for immune intervention.

### OSKM-Expressing CSC-Like Cells as a Source of Tumor Antigens

The main objective of the present study was to investigate whether human DCs pulsed with OSKM-expressing CRC cells could induce Sox2/Oct4-specific CTLs and colorectal CSC antigen-specific CTLs. These CRC cells have OSKM gene products as over-expressed “tumor-associated” antigens, which are expressed at high densities on cell surface. Pluripotency-associated antigens are attractive as cancer immunotherapy targets, since this class of antigens may be less susceptible to immune tolerance mechanisms that may limit the repertoire of reactive T cells, especially the high-avidity T cells, present in vivo. Clinically, vaccines targeting pluripotency-associated genes on CSCs could be useful against diverse cancers with poorly differentiated characteristics, instead of targeting a defined subset of cancer. Thus, these vaccines may serve as a universal cancer immunotherapeutic platform, particularly in the setting of maintenance therapy to prevent or delay cancer from returning. The ability of the human immune system to mediate T cell responses against pluripotency-associated genes has been investigated. When human leucocyte antigen (HLA)-A*0201-restricted Sox2-derived peptides are tested for the activation of glioma-reactive CD8+ CTLs, specific CTLs against the peptide can be raised and are capable of lysing glioma cells, confirming Sox2 as a target antigen for CTLs. While Oct4-specific memory CD4+ T cells are readily detectable in peripheral blood of healthy humans, immunity to Oct4 was detected in only 35% of patients with newly diagnosed germ-cell tumors.

However, chemotherapy of germ-cell tumors leads to the induction of anti-Oct 4 immunity in vivo in these patients, demonstrating the lack of immune tolerance to Oct4 in humans. Besides pluripotency-associated genes, OSKM-engineered CRC cells express many CSC-associated tumorigenic antigens and hence serve as a rich source of CSC antigens that arise as a result of the OSKM gene-mediated reprogramming Since they also carry original somatic mutation, internal deletions, chromosome translocation in the parent cancer cells and many unidentified neoantigens associated with the changes, these OSKM-expressing CRC cells will provide a broad spectrum of tumor antigens.

The use of a stable cancer cell line provides many advantages for vaccine preparation and application. Firstly, the use of an established cell line helps to circumvent a time-consuming and cost intensive patient-individualized GMP production and eliminates the need for the continuous production of tailor-made individual vaccines. Secondly, the use of an established cell line simplifies the logistics, reduces the laboriousness of the vaccine production and delivery process, and increases its cost effectiveness. Thirdly, the use of an established cell line allows for the highly standardized and large scale production of allogeneic vaccines, so called “off-the-shelf” products suitable for all patients with a particular tumour type. Lastly, the use of a single batch of allo-vaccines for all vaccines, independent of HLA haplotype, eliminates variability in the quality and composition of the vaccines, facilitating reliable comparative analysis of clinical outcome.

### CONCLUSION

After primary tumor resection, median survival is 8 months in patients with stage IV CRC who did not receive chemotherapy and 21 months in the patients who received chemotherapy. Active chemotherapeutical drugs such as irinotecan (CPT-11) and oxaliplatin (OHP) have been introduced to treat patients with metastatic CRC. However, limited by side effects and cumulative toxicities, the length of continuous chemotherapy rarely exceeds 6 months. As a classical way, chemotherapy is performed with a pre-defined number of cycles (3-6 months), followed by complete break or maintenance with less toxic drugs. The current study has the potential to lead to the development of an autologous immune cell-based treatment regimen, namely DC-based immunotherapy targeting CSCs, with the hope that the regimen can be used as an option for CRC maintenance therapy. While several laboratories have tested DC cancer immunotherapy for CRC, an effort of using DC approaches to target CSCs is still a pioneering work that has not been explored before. The current study is attractive as it capitalizes on a genetic engineering approach to enhance CSC properties of CRC cells before using them for DC pulsing. The findings disclosed herein on the transcriptional regulation of CSC gene expression by OSKM factors have not only unraveled the fundamental understanding on molecular regulatory mechanisms in CSCs, but also provide important leads for the development of new CRC therapeutics.

Supplementary Table 1
Primers for targeted OKSM transgene integration and over-expression

| Primer Sequence (5′ to 3′) | SEQ | Amplicon | |
|---|---|---|---|
| (Forward, FP; Reverse, RP) | ID NO: | Size (bp) | Description |
| FP: TATTCAGCCAAACGACCATC | 1 | 197 | Quantify total Oct4 |
| RP: GCCTCTCACTCGGTTCTC | 2 | | expression |
| FP: ACGACGTGAGCGCCCTGCAGTACAA | 3 | 155 | Quantify total Sox2 |
| RP: GCTGGAGCTGGCCTCGGACTTGACC | 4 | | expression |
| FP: CCTACACAAAGAGTTCCCATC | 5 | 123 | Quantify total Klf4 |
| RP: AGTGCCTGGTCAGTTCATC | 6 | | expression |
| FP: AGGAACAAGAAGATGAGGAAG | 7 | 170 | Quantify total c-Myc |
| RP: TGCGTAGTTGTGCTGATG | 8 | | expression |
| FP: GTACTCCTCGGTCCCTTTCC | 9 | 143 | Quantify exogenous Oct4 |
| RP: CACCTGCAAGTTTCAGCAAA | 10 | | expression |
| FP: CATGTCCCAGCACTACCAGA | 11 | 123 | Quantify exogenous Sox2 |
| RP: ACATCCCCTGCTTGTTTCAA | 12 | | expression |
| FP: GACCACCTCGCCTTACACAT | 13 | 138 | Quantify exogenous Klf4 |
| RP: CCAAAAGACGGCAATATGGT | 14 | | expression |
| FP: AAGAGGACTTGTTGCGGAAA | 15 | 182 | Quantify exogenous c- |
| RP: GGCATTAAAGCAGCGTATCC | 16 | | Myc expression |
| FP: AAGGAATTGGGAACACAAAGG | 17 | 83 | Quantify endogenous |
| RP: CAAGAGCATCATTGAACTTCAC | 18 | | Oct4 expression |
| FP: GGGAAATGGGAGGGGTGCAAAAGAGG | 19 | 151 | Quantify endogenous |
| RP: TTGCGTGAGTGTGGATGGGATTGGTG | 20 | | Sox2 expression |
| FP: TGGTGCTTGGTGAGTCTTG | 21 | 117 | Quantify endogenous Klf4 |
| RP: AGGTCATAAATGTTGATCGGAAG | 22 | | expression |
| FP: CCTTGCCGCATCCACGAAAC | 23 | 77 | Quantify endogenous c- |
| RP: CCTTGCTCGGGTGTTGTAAGTTC | 24 | | Myc expression |
| ACAGAATTCCTGCGCGCCAAGTTACGGCGCGCCC | 25 | 63 | Adaptor sequence bearing |
| TTAGCATACATTATACCTGCAGGCAC | 26 | | EcoRI-AscI-SbfI restriction |
| | | | sites |
| FP: GCTACGTCCCTTCGGCCCTCAATC | 27 | 3038 | Check for site-specific |
| RP: GCCTCCCTAAGACCCAGAAGTCCAG | 28 | | integration of OKSM- |
| | | | eGFP donor at AAVS1 |
| | | | locus |
| FP: GACTGGTATTCTTAACTATGTTGCTCC | 29 | 538 | Probe design for Southern |
| RP: CAAAGGGAGATCCGACTCGTCTGA | 30 | | blot to detect multiple |
| | | | integration of OKSM- |
| | | | eGFP cassette in genome |

Supplementary Table 1 (continued)
Primers for DNA methylation analysis

| Primer Sequence (5′ to 3′) | SEQ | Amplicon | |
|---|---|---|---|
| (Forward, FP; Reverse, RP) | ID NO: | Size (bp) | Description |
| FP: GAGGTTGGAGTAGAAGGATTGTTTTGGTTT | 31 | 467 | Amplify bisulfide- |
| RP: CCCCCCTAACCCATCACCTCCACCACCTAA | 32 | | treated Oct3/4 |
| | | | promoter |
| FP: GAGGCTGGAGCAGAAGGATTGCTTTGGCCC | 33 | 467 | Amplify bisulfide |
| RP: CCCCCCTGGCCCATCACCTCCACCACCTGG | 34 | | untreated Oct3/4 |
| | | | promoter |
| FP: TGGTTAGGTTGGTTTTAAATTTTTG | 35 | 335 | Amplify bisulfide- |
| RP: AACCCACCCTTATAAATTCTCAATTA | 36 | | treated Nanog |
| | | | promoter |
| FP: TGGCCAGGCTGGTTTCAAACTCCTG | 37 | 335 | Amplify bisulfide |
| RP: GACCCACCCTTGTGAATTCTCAGTTA | 38 | | untreated Nanog |
| | | | promoter |
| FP: GGTTTAAAAGGGTAAATGTGATTATATTTA | 39 | 360 | Amplify bisulfide- |
| RP: CAAACTACAACCACCCATCAAC | 40 | | treated Rex1 |
| | | | promoter |
| FP: GGCCTAAAAGGGTAAATGTGATTACACCCA | 41 | 360 | Amplify bisulfide |
| RP: CAGGCTACAGCCACCCATCAGC | 42 | | untreated Rex1 |
| | | | promoter |

Supplemtary Table 1 (continued)
Primers for human colorectal cancer stem cell markers

| Primer Sequence (5′ to 3′) | SEQ | Amplicon | |
|---|---|---|---|
| (Forward, FP; Reverse, RP) | ID NO: | Size (bp) | Description |
| FP: CTTCATCCACAGATGCTCCTAAG | 43 | 191 | Amplify mRNA |
| RP: TGGATTCATATGCCTTCTGTAAGA | 44 | | CD133 |
| FP: AGGGATCCTCCAGCTCCTTTCG | 45 | 191 | Amplify mRNA |
| RP: CGTCCGAGAGATGCTGTAGCGA | 46 | | CD44 |
| FP: GTGTGTCTGGGAGAAGACGCTG | 47 | 270 | Amplify mRNA |
| RP: GGTACGTCAAGTCGGCAAGGTATG | 48 | | CD166 |
| FP: ATCCACAAGCCAGACTAGAAGGC | 49 | 278 | Amplify mRNA |
| RP: AGATCCAACTGCTTCCTTTCTCAG | 50 | | ABC B5 |
| FP: TGAGCCAGTCACCTGTGTTCCA | 51 | 188 | Amplify mRNA |
| RP: TGGCAGAGCTCCTCCTCAGTTG | 52 | | ALDH1A1 |
| FP: ATCAGACGCGCAGAGGAGGC | 53 | 281 | Amplify mRNA |
| RP: GAGCAAACACACAGCAAACTGAACTG | 54 | | CD29 |
| FP: ACGGAGGAAGGTCTGAGGAGCA | 55 | 170 | Amplify mRNA |
| RP: TGAGTAGCCATTGTCCACGCTGG | 56 | | CTNNB1 |
| FP: ACAGTGCGGCAGACGTAAGGAT | 57 | 110 | Amplify mRNA |
| RP: GGAGCAGCTGACTGATGTTGTTCATAC | 58 | | LGR5 |
| FP: GTGGAAGGTTCTTCTGGGACT | 59 | 322 | Amplify mRNA |
| RP: ACTGCCCATCAGGAGATATTGAAT | 60 | | CD26 |
| FP: CATTCACTTTTGAAAAGTTCTCCCTAGAAAG | 198 | 272 | Amplify mRNA |
| RP: CGTTTTTGTGCAGTTTTAAAATGTGTGC | 199 | | DPP4 |
| FP: GGTTCTCATATGAACCTAACTGGTCAAA | 200 | 258 | Amplify mRNA |
| RP:ATCATGTTTATAAAAGCAATAGAAGGTACGGT | 201 | | ITGB1 |
| FP: AAGGTGATCTATTTTTCCCTTTCTCC | 202 | 171 | Amplify mRNA KIT |
| RP: TTTCATACTGACCAAAACTCAGCCT | 203 | | |
| FP: GACTCCTACAACCCGAATACTGC | 204 | 242 | Amplify mRNA MET |
| RP: CATAGTGCTCCCCAATGAAAGTAGAG | 205 | | |

Supplementary Table 1 (continued)
Primers for epithelial-mesenchymal transition (EMT) markers

| Primer Sequence (5′ to 3′) | SEQ | Amplicon | |
|---|---|---|---|
| (Forward, FP; Reverse, RP) | ID NO: | Size (bp) | Description |
| FP: GGGTGACTACAAAATCAATC | 61 | 252 | Amplify mRNA E-cadherin |
| RP: GGGGGCAGTAAGGGCTCTTT | 62 | | |
| FP: TGAAGGAGGAAATGGCTCGTC | 63 | 137 | Amplify mRNA Vimentin |
| RP: GTTTGGAAGAGGCAGAGAAATCC | 64 | | |
| FP: GGAGTTTCCTGAGGGTTT | 65 | 208 | Amplify mRNA Fibronectin |
| RP: GCAGAAGTGTTTGGGTGA | 66 | | |
| FP: CGAATGGATGAAAGACCCATCC | 67 | 174 | Amplify mRNA N-cadherin |
| RP: GGAGCCACTGCCTTCATAGTCAA | 68 | | |
| FP: ATCGGAAGCCTAACTACAGCGAGC | 69 | 150 | Amplify mRNA Snail |
| RP: CAGAGTCCCAGATGAGCATTGG | 70 | | |
| FP: GGAGTCCGCAGTCTTACGAG | 71 | 201 | Amplify mRNA Twist |
| RP: TCTGGAGGACCTGGTAGAGG | 72 | | |
| FP: ACCCTTGAAAGTGATCCAGC | 73 | 142 | Amplify mRNA ZEB1 |
| RP: CATTCCATTTTCTGTCTTCCGC | 74 | | |
| FP: AGATGCATATTCGGACCCAC | 75 | 258 | Amplify mRNA Slug |
| RP: CCTCATGTTTGTGCAGGAGA | 76 | | |
| FP: ATTCCTGGCGATACCTCAGC | 77 | 186 | Amplify mRNA TGFb1 |
| RP: ACCCGTTGATGTCCACTTGC | 78 | | |
| FP: GGCCTCGAGCTGGGAATCGC | 79 | 100 | Amplify mRNA Sox4 |
| RP: GCCCACTCGGGGTCTTGCAC | 80 | | |

Supplementary Table 1 (continued)
Primers for colorectal cancer prognosis biomarkers

| Primer Sequence (5′ to 3′) | SEQ | Amplicon | |
|---|---|---|---|
| (Forward, FP; Reverse, RP) | ID NO: | Size (bp) | Description |
| FP: AACTTCTCCTGGTCTCTCAGCT | 81 | 145 | Amplify mRNA CEA |
| RP: GCAAATGCTTTAAGGAAGAAG | 82 | | |
| FP: CAAACCAAGCCCAGAACCTG | 83 | 153 | Amplify mRNA B3GALT5 (CA |
| RP: TCAATCTCATCTTCGGGAAAGC | 84 | | 19-9) |
| FP: GCCGGAGATGAGGAAACTGA | 85 | 180 | Amplify mRNA ST6GALNAC6 |
| RP: GATCACGAACACTGCTGACC | 86 | | (CA 19-9) |
| FP: ACCTGAATCACATCGAGCCA | 87 | 144 | Amplify mRNA TS |
| RP: TTGGATGCGGATTGTACCCT | 88 | | |
| FP: CGCCTGGTGACTTCTCC | 89 | 238 | Amplify mRNA TP |
| RP: TGGGTCAGCACCGAGGT | 90 | | |
| FP: GTTGTGGCTATGATTGATGA | 91 | 237 | Amplify mRNA DPD |
| RP: ATTCACAGATAAGGGTACGC | 92 | | |
| FP: TTCCGCCATGGTTTTTAAATCA | 93 | 233 | Amplify mRNA DCC |
| RP: AGCCTCATTTTCAGCCACACA | 94 | | |
| FP: ACTGGGGAGGGCTTTCTTTG | 95 | 246 | Amplify mRNA KRAS |
| RP: GGCATCATCAACACCCTGTCT | 96 | | |
| FP: GGTGGTGCCCTATGAGCCG | 97 | 210 | Amplify mRNA TP53 |
| RP: TCCTCTGTGCGCCGGTCTC | 98 | | |
| FP: CCAATCAAAGGAGGGCTCACC | 99 | 385 | Amplify mRNA GPA33 |
| RP: TTCTCTTAGCTGCTCTGGTGGC | 100 | | |

Supplemetary Table 1 (continued)
Primers for colorectal cancer top risk loci identified GWASs

| Primer Sequence (5′ to 3′) | SEQ | Amplicon | |
|---|---|---|---|
| (Forward, FP; Reverse, RP) | ID NO: | Size (bp) | Description |
| FP: TGGCTCGCATTCATTTTCTG | 101 | 309 | Amplify mRNA BMI-1 |
| RP: AGTAGTGGTCTGGTCTTGTG | 102 | | |
| FP: GGAAGCAGAGAAAGTACTGGA | 103 | 114 | Amplify mRNA APC |
| RP: CTGAAGTTGAGCGTAATACCAG | 104 | | |
| FP: CTGCTGCTGGAATTGGTGTTGA | 105 | 146 | Amplify mRNA SMAD4 |
| RP: CTGGAGGGCCCGGTGTAAGT | 106 | | |
| FP: CTGGCTCCAGAAGATCACAAAG | 107 | 239 | Amplify mRNA AXIN2 |
| RP: ATCTCCTCAAACACCGCTCCA | 108 | | |
| FP: GACTACACGGGAGCCACTGTCA | 109 | 117 | Amplify mRNA POLD1 |
| RP: GTAACACAGGTTGTGGGCCATC | 110 | | |
| FP: GAAGTCGGAACACAAGGAAG | 111 | 395 | Amplify mRNA STK11 |
| RP: CCGTAACCTCCTCAGTAGTT | 112 | | |
| FP: GTCAGAAAATGGAGTAACCTTA | 113 | 560 | Amplify mRNA BMPR1A |
| RP: TAGTTCGCTGAACCAATAAAGG | 114 | | |
| FP: GGCCTCTGTCTCCCCATATCAT | 115 | 397 | Amplify mRNA MUTYH |
| RP: CTGCTGTAGGGTCTCTGCTGTA | 116 | | |
| FP: CTGCAACCCCCATCACCTTA | 117 | 133 | Amplify mRNA SMAD7 |
| RP: CCCTGTTTCAGCGGAGGAA | 118 | | |
| FP: GGTGGAATGACTGGATTG | 119 | 189 | Amplify mRNA BMP2 |
| RP: GCATCGAGATAGCACTG | 120 | | |
| FP: CTGGTCCACCACAATGTGAC | 121 | 162 | Amplify mRNA BMP4 |
| RP: CGATCGGCTAATCCTGACAT | 122 | | |
| FP: GAGAACGACGGCTACTTTCGGA | 123 | 227 | Amplify mRNA RHPN2 |
| RP: AGCTCTTCCTTGAGCATCTGCA | 124 | | |
| FP: ATATCCAGCTCACCAGGCCATGAA | 125 | 131 | Amplify mRNA SCG5 |
| RP: TGTTGTCTCCAGTCAACTCTGCCA | 126 | | |
| FP: GTCACACTCAACTGCCCTGA | 127 | 375 | Amplify mRNA GREM1 |
| RP: GGTGAGGTGGGTTTCTGGTA | 128 | | |
| FP: CAGCATCTCTGTTCCTCCATCA | 129 | 271 | Amplify mRNA FMN1 |
| RP: AGTGCCTTCCATTATGCCTACC | 130 | | |

Supplementary Table 1 (continued)
Primers for transcriptional regulation of CD24 core promoter

| Primer Sequence (5′ to 3′) | SEQ | Amplicon | |
|---|---|---|---|
| (Forward, FP; Reverse, RP) | ID NO: | Size (bp) | Description |
| FP (XbaI): GCGCTCTAGACTGCAGGGATTAGCGCCTGGAG | 131 | 2.4k | Construct PGK-Luc- |
| RP (FseI): | 132 | | Intron to examine intron |
| ATATGGCCGGCCCCACATCACAGCTACCTCCATGTAC | | | 2 (2.4 kb) transcriptional |
| | | | regulation activity of |
| | | | CD24 |
| FP (EcoRV): GATGATATCGATACCAGCCGGGTACCAGCAG | 133 | 0.26k | Construct CD24-derived |
| RP (HindIII): GATAAGCTTTCAGGATGCTGGGTGCTTGGAG | 134 | | luciferase reporter to |
| examine core promoter | | | |
| activity of CD24 | | | |
| FP (EcoRV): GATGATATCGCAGTCTGAGTGGCAATGCACTTG | 135 | 0.5k | Construct CD24-derived |
| RP (HindIII): GATAAGCTTTCAGGATGCTGGGTGCTTGGAG | 136 | | luciferase reporter to |
| examine core promoter | | | |
| activity of CD24 | | | |
| FP (EcoRV): GATGATATCCCTGTAGTTTGCAGCGTCAGGCA | 137 | 0.9k | Construct CD24-derived |
| RP (HindIII): GATAAGCTTTCAGGATGCTGGGTGCTTGGAG | 138 | | luciferase reporter to |
| examine | | | core promoter |
| activity of CD24 | | | |
| FP (EcoRV): GATGATATCCCACGCCCGGCCAAAGTATTTC | 139 | 1.9k | Construct CD24-derived |
| RP (HindIII): GATAAGCTTTCAGGATGCTGGGTGCTTGGAG | 140 | | luciferase reporter to |
| | | | examine core promoter |
| | | | activity of CD24 |

Supplementary Table 1 (continued)
Biotin-labelled oligonucleotides for EMSA assay

| Primer Sequence (5′ to 3′) | SEQ | Oligo | Description |
|---|---|---|---|
| (Forward, FP; Reverse, RP) | ID | Size | |
| FP: TGGCAGGTCCCGGGAAACAAAGGAAACTTGGGCCCGGC | 141 | 38 | CD24 promoter region |
| RP: GCCGGGCCCAAGTTTCCTTTGTTTCCCGGGACCTGCCA | 142 | | bearing a Sox2 binding |
| | | | site |
| FP: CCGGGAAACAAAGGAAACTCCGGGAAACAAAGGAAACT | 143 | 38 | CD24 promoter region |
| RP: AGTTTCCTTTGTTTCCCGGAGTTTCCTTTGTTTCCCGG | 144 | | bearing two Sox2 |
| | | | binding sites |
| FP: CCAGCCGGGTACCAGCAGCCGGCGGCGCCCGCCCACCT | 145 | 38 | CD24 promoter region |
| RP: AGGTGGGCGGGCGCCGCCGGCTGCTGGTACCCGGCTGG | 146 | | bearing no Sox2 |
| | | | binding site (none |
| | | | relevant sequence 1) |
| FP: GCTTTCCTGGTATATAAGGTCTCGCCGGCTCGCCGCGC | 147 | 38 | CD24 promoter region |
| RP: GCGCGGCGAGCCGGCGAGACCTTATATACCAGGAAAGC | 148 | | bearing no Sox2 |
| | | | binding site (none |
| | | | relevant sequence 2) |
| FP: TGGCAGGTCCCGGGAAAAGGAGGAAACTTGGGCCCGGC | 149 | 38 | CD24 promoter region |
| RP: GCCGGGCCCAAGTTTCCTCCTTTTCCCGGGACCTGCCA | 150 | | bearing a mutated Sox2 |
| | | | binding site |
| FP: TGGCAGGTCCATTTGCATAACAAAGAGTTGGGCCCGGC | 151 | 38 | CD24 promoter region |
| RP: GCCGGGCCCAACTCTTTGTTATGCAAATGGACCTGCCA | 152 | | bearing a consensus |
| | | | Sox2 binding site |
| | | | (positive control) |

Supplementary Table 1 (continued)
Primers for knockout of miR-205 binding site on CD24 3′UTR

| Primer Sequence (5′ to 3′) | SEQ | Amplicon | |
|---|---|---|---|
| (Forward, FP; Reverse, RP) | ID NO: | Size (bp) | Description |
| FP: AAACGAGTTTCATGTACAAGATGAGT | 153 | — | Construct CRISPR to |
| RP: TAAAACTCATCTTGTACATGAAACTC | 154 | | knockout miR205 |
| | | | binding site on CD24 3′- |
| | | | UTR |
| FP: CAGTCAACAGCCAGTCTCTTCGTG | 155 | 714 | T7E1 assay to quantify |
| RP: CATCATTGCCCTGGCACATGTCA | 156 | | CRISPR-mediated |
| | | | cleavage efficiency on |
| | | | targeted CD24 site |
| FP (SnaBI): GCGCTACGTACCAGGATAGACAGTGACCCAATGA | 157 | 867 | Construct left |
| RP (SaII): ATATGTCGACCTTGTACATGAAACTCCAGCAGAT | 158 | | homologous arm bearing |
| | | | CD24 sequences |
| FP (NotI): ATATGCGGCCGCGAAGGAGAGGCAACATCCAAAATAG | 159 | 875 | Construct right |
| RP (BstBI): GCGCTTCGAATCAAGCCTGTAATCCCAGCACTTTG | 160 | | homologous arm bearing |
| | | | CD24 sequences |
| FP: GACATCACCTCCCACAACGAGGACT | 161 | 1.85k | Check for site-specific |
| RP: AGCATCAGTGTGTGACCATGCGAAC | 162 | | integration of mCherry |
| | | | transgene on CD24 |
| FP: GAGCAATGGTGGCCAGGCTC | 163 | 290 | Amplify mRNA CD24 on |
| RP: GGATTGGGTTTAGAAGATGGGGAAA | 164 | | chromosome 6 |
| FP: GAGCAATGGTGGCCAGGCTT | 165 | 290 | Amplify mRNA CD24 on |
| RP: GGATTGGGTTTAGAAGATGGGGAAG | 166 | | chromosome Y |
| FP: ATCCAGCCAGACACGCTGCA | 167 | 187 | Amplify mRNA CD24 on |
| RP: CAGTAGCTGGATTTGGGGCAGTC | 168 | | chromosome 15 |
| FP: TCTGGAAGTCCAATGTGGCAAG | 169 | 406 | Amplify truncated mRNA |
| RP: CACTGGAAGTTCCCTTCTCATGTAC | 170 | | CD24 expressed by |
| | | | chromosome 6 |
| FP: AGATCCTCAGACAATCCATGTGCT | 171 | 104 | Quantify precursor stem- |
| RP: AGCTCCATGCCTCCTGAACT | 172 | | loop hsa-mir-205 |
| | | | expression |
| Adaptor: TCCGTCGTTCTAATGCGAACAGACTCCG | 173 | — | cDNA synthesis of |
| | | | mature miR205-5p |
| FP: CCTCCATCCTTCATTCCACCG | 174 | 50 | Quantify mature |
| RP: GTTTCCGTCGTTCTAATGCGAA | 175 | | miR205-5p expression |
| FP: CTCAAGTACCCATCTTGGAGGG | 176 | 244 | Quantify miR205HG |
| RP: CACGCACACTCCAGATGTCTC | 177 | | expression |
| FP (EcoRI): GCGCGAATTCAAAGATCCTCAGACAATCCATGTGCT | 178 | 114 | Construct CMV-miR205 |
| RP (SpeI): ATATACTAGTTGTCAGCTCCATGCCTCCTGAAC | 179 | | |
| FP (EcoRI): GCGCGAATTCGAAATGGGCTGAGTCCCTCTTG | 180 | 963 | Construct CMV- |
| RP (SpI): ATATACTAGTTTTTTCAGTAGACAAGCAACTTTTAG | 181 | | miR205HG (co-expressing miR205) |
| FP (XbaI): GCGCTCTAGACTTAAGAGACTCAGGCCAAGAAACG | 182 | 211 | Construct PGK-Luc-UTR |
| RP (FseI): ATATGGCCGGCCGGCATCCATCATCTAGTCAAACCTC | 183 | | to examine 3′-UTR |
| | | | (211 bp) transcriptional |
| | | | regulation activity of |
| | | | CD24 |
| FP (XbaI): GCGCTCTAGACTTAAGAGACTCAGGCCAAGAAACG | 184 | 300 | Construct PGK-Luc-UTR |
| RP (FseI): ATATGGCCGGCCCAAGCCACATTCAAGGAAATCATGTCT | 185 | 301 | to examine 3′-UTR |
| | | 514 | transcriptional regulation |
| | | | activity of CD24 |

Supplemtary Table 1 (continued)
Primers for knockout of CD24 ORF

| Primer Sequence (5′ to 3′) | SEQ | Amplicon | |
|---|---|---|---|
| (Forward, FP; Reverse, RP) | ID NO: | Size (bp) | Description |
| FP: AAACGGACATGGGCAGAGCAATGGGT | 186 | — | Construct CRISPR to |
| RP: TAAAACCCATTGCTCTGCCCATGTCC | 187 | | knockout CD24 ORF |
| FP: CACGTCACGGCTATTGTGGCTTTC | 188 | 532 | T7E1 assay to quantify |
| RP: GCCTCTGGGTGAAAGTGGGAAGTAG | 189 | | cCRISPR-mediated leavage |
| | | | efficiency on targeted CD24 |
| | | | site |
| FP (SnaBI): GCGCTACGTAGCTCACAGAACAAAGCAAGGGCTTC | 190 | 881 | Construct left homologous |
| RP (SaII): ATATGTCGACTTGCTCTGCCCATGTCCCCT | 191 | | arm bearing CD24 |
| | | | sequences |
| FP (NotI): ATATGCGGCCGCTGGTGGCCAGGCTCGGGCT | 192 | 824 | Construct right homologous |
| RP (BstBI): GCGCTTCGAAAGGATCTAGGGAGACCGCGCTGGTAG | 193 | | arm bearing CD24 |
| | | | sequences |
| FP: GACATCACCTCCCACAACGAGGACT | 194 | 1.6k | Check for site-specific |
| RP: ATCCTCCAAACCCGAACTGACCCA | 195 | | integration of mCherry |
| | | | transgene on CD24 |
| FP: GAGCAATGGTGGCCAGGCTC | 196 | 290 | Amplify mRNA CD24 |
| RP: GGATTGGGTTTAGAAGATGGGGAAA | 197 | | |

### REFERENCES

1. Hockemeyer, D., Soldner, F., Beard, C., Gao, Q., Mitalipova, M., DeKelver, R. C., Katibah, G. E., Amora, R., Boydston, E. A., Zeitler, B. et al. (2009) Efficient targeting of expressed and silent genes in human ESCs and iPSCs using zinc-finger nucleases. Nature biotechnology, 27, 851-857.
2. Miller, J. C., Holmes, M. C., Wang, J., Guschin, D. Y., Lee, Y. L., Rupniewski, I., Beausejour, C. M., Waite, A. J., Wang, N. S., Kim, K. A. et al. (2007) An improved zinc-finger nuclease architecture for highly specific genome editing. Nature biotechnology, 25, 778-785.
3. Phang R Z, Tay F C, Goh S L, Lau C H, Zhu H, Tan W K, Liang Q, Chen C, Du S, Li Z, Tay J C, Wu C, Zeng J, Fan W, Toh H C, Wang S. Zinc finger nuclease-expressing baculoviral vectors mediate targeted genome integration of reprogramming factor genes to facilitate the generation of human induced pluripotent stem cells. Stem Cells Transl Med. 2013 December; 2(12):935-45.
4. Ramachandra, C. J., Shahbazi, M., Kwang, T. W., Choudhury, Y., Bak, X. Y., Yang, J. and Wang, S. (2011) Efficient recombinase-mediated cassette exchange at the AAVS1 locus in human embryonic stem cells using baculoviral vectors. Nucleic Acids Research, 39, e107.
5. Tay F C, Tan W K, Goh S L, Ramachandra C J, Lau C H, Zhu H, Chen C, Du S, Phang R Z, Shahbazi M, Fan W, Wang S. Targeted transgene insertion into the AAVS1 locus driven by baculoviral vector mediated zinc finger nuclease expression in human-induced pluripotent stem cells. J Gene Med. 2013 October; 15(10):384-95.
6. Zhu, H., Lau, C. H., Goh, S. L., Liang, Q., Chen, C., Du, S., Phang, R. Z., Tay, F. C., Tan, W. K., Li, Z. et al. (2013) Baculoviral transduction facilitates TALEN-mediated targeted transgene integration and Cre/LoxP cassette exchange in human-induced pluripotent stem cells. Nucleic acids research, 41, e180.

1.-41. (canceled)

42. A composition comprising at least one isolated dendritic cell loaded with at least one colorectal cancer stem cell, or fragments thereof, wherein the colorectal cancer stem cell comprises a differentiated colorectal tumour cell that has been reprogrammed with a baculoviral vector into an undifferentiated colorectal stem-cell state, wherein the baculoviral vector comprises zinc-finger nuclease-coding sequences and a fusion gene comprising a cell reprogramming factor or transcription factor capable of reverting or reprogramming the differentiated colorectal tumour cell into the undifferentiated colorectal cancer stem-cell state.

43. The composition of claim 42, wherein the dendritic cell is a mature dendritic cell.

44. The composition of claim 43, wherein the mature dendritic cell comprises an immature dendritic cell that has been loaded with said at least one colorectal cancer stem cell, or fragments thereof.

45. The composition of claim 44, wherein the mature dendritic cell has upregulated CD83, CD40 and CD86 expression.

46. The composition of claim 42, wherein the colorectal cancer stem cell is an in vitro (test-tube) colorectal stem cell that has been reprogrammed in vitro.

47. The composition of claim 42, wherein the colorectal cancer stem cell is a heat-shocked colorectal cancer stem cell, or fragments thereof.

48. The composition of claim 47, wherein the fragment is a lysate of a heat-shocked colorectal cancer stem cell.

49. The composition of claim 42, wherein the cell reprogramming factor or transcription factor is selected from the group consisting of Oct4, Sox2, Klf4 and c-myc.

50. A composition for stimulating the immune system of a subject comprising at least one dendritic cell loaded with one or more antigens of a colorectal cancer stem cell, wherein said colorectal cancer stem cell comprises a differentiated colorectal tumour cell that has been reprogrammed with a baculoviral vector into an undifferentiated colorectal stem-cell state, wherein the baculoviral vector comprises zinc-finger nuclease-coding sequences and a fusion gene comprising a cell reprogramming factor or transcription factor selected from the group consisting of Oct4, Sox2, Klf4 and c-myc.

51. The composition of claim 50, wherein both the dendritic cell and the colorectal tumour cell are from the same or different subject.

52. The composition claim 42, wherein the undifferentiated colorectal stem-cell state is characterised by at least one of the following:
(a) loss of epithelial characteristics with reduction in E-cadherin expression,
(b) gain of mesenchymal properties with differential expression of vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and SOX4; and
(c) the expression of cancer stem cell markers selected from the group consisting of CD24, CD133, CD44, CD166, aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1).

53. The composition of claim 50, wherein the undifferentiated colorectal stem-cell state is characterised by at least one of the following:
(a) loss of epithelial characteristics with reduction in E-cadherin expression,
(b) gain of mesenchymal properties with differential expression of vimentin (VIM), fibronectin (FN1), vitronectin (VTN), N-cadherin (CDH2), snail (SNAI1), twist (TWIST1), zinc finger E-box-binding homeobox 1 (ZEB1), transforming growth factor beta 1 (TGFB1), slug (SNAI2) and SOX4; and
(c) the expression of cancer stem cell markers selected from the group consisting of CD24, CD133, CD44, CD166, aldehyde dehydrogenase 1 (ALDH1A1), leucine-rich repeat-containing G-protein coupled receptor 5 (LGR5), dipeptidyl peptidase 4 (DPP4), catenin beta-1 (CTNNB1), ATP-binding cassette sub-family G member 5 (ABCG5) and integrin beta-1 (ITGB1).

54. A composition for pulsing a dendritic cell such that the dendritic cell is capable of inducing specific immune response of cytotoxic T lymphocytes against an in vitro colorectal cancer stem cell, said composition comprising at least one in vitro colorectal cancer stem cell-like cell, or fragments thereof, which has been enriched from an in vitro colorectal cancer cell that has been reprogrammed with a baculoviral vector into an undifferentiated colorectal stem-cell state, wherein the baculoviral vector comprises zinc-finger nuclease-coding sequences and a fusion gene comprising a cell reprogramming factor or transcription factor capable of reverting or reprogramming the differentiated colorectal tumour cell into the undifferentiated colorectal cancer stem-cell state.
The composition of claim 13, wherein the inducing of the specific immune response comprises presenting antigens derived from said in vitro colorectal cancer stem cell to a population of T cells and inducing differentiation of said population of T cells into said cytotoxic T lymphocytes, wherein said cytotoxic T lymphocytes are specific against said antigens.

55. The composition of claim 54, wherein the inducing of the specific immune response comprises presenting antigens derived from said in vitro colorectal cancer stem cell to a population of T cells and inducing differentiation of said population of T cells into said cytotoxic T lymphocytes, wherein said cytotoxic T lymphocytes are specific against said antigens.
